(19) United States
(12) Patent Application Publication
(10) Pub. No.: US 2011/0197294 A1
(43) Pub. Date: Aug. 11, 2011

(54) COMPOSITIONS AND METHODS FOR RESTORING MITOCHONDRIAL ELECTRON TRANSFER FUNCTION

(76) Inventors: Roberta A. Gottlieb, San Diego, CA (US); Cynthia Perry, San Diego, CA (US)

(21) Appl. No.: 12/995,993

(22) PCT Filed: Jun. 4, 2009
(86) PCT No.: PCT/US2009/046332

§ 371 Date: Mar. 8, 2011

Related U.S. Application Data
(60) Provisional application No. 61/058,868, filed on Jun. 4, 2008.

(51) Int. Cl. A01K 67/027; C12N 9/02; C12N 9/96; C07H 21/00; A61K 38/44; A61K 9/127; C12N 15/63; C12N 5/10; C12N 1/21; C12N 1/15; C12N 1/19; A61P 9/10; A61P 9/00; A61P 25/28; A61P 25/16; A61P 43/00; A61P 3/04; A61P 9/04

(52) U.S. Cl. 800/18; 435/189; 435/188; 536/23.2; 424/94.4; 424/450; 424/94.3; 435/320.1; 435/325; 435/252.3; 435/254.11; 435/254.2; 435/419; 435/348; 800/14

(57) ABSTRACT

The invention provides methods and compositions for treating, ameliorating or preventing diseases or conditions caused by or aggravated by lost and/or impaired mitochondrial Complex I function, including treating, ameliorating or preventing an ischemia and/or reperfusion injury, Parkinson's disease, myopathic diseases, cardiolipin deficiency, neurodegenerative diseases, aging, diabetes, obesity, sepsis and other conditions in which mitochondrial Complex I function is lost and/or impaired.

### TECHNICAL FIELD

This invention relates to medicine, cellular biology and biochemistry. In one aspect, the invention provides methods and compositions for treating, ameliorating or preventing diseases or conditions caused by or aggravated by lost and/or impaired mitochondrial Complex I function, including treating, ameliorating or preventing an ischemia and/or reperfusion injury, Parkinson's disease, myopathic diseases, cardiolipin deficiency, neurodegenerative diseases, aging, diabetes, obesity, and other conditions in which mitochondrial Complex I function is lost and/or impaired.

### BACKGROUND

The NADH:ubiquinone oxidoreductase, or complex I of the mitochondrial respiratory chain, is an enzyme with a vital role in energy metabolism, where mutations affecting complex I can affect at least three processes: impair the oxidation of NADH; reduce the enzyme's ability to pump protons for the generation of a mitochondrial membrane potential; and, increase the production of damaging reactive oxygen species.

Mitochondrial dysfunction, such as defects in the NADH-quinone oxidoreductase (complex I), is recognized as closely related to the etiology of sporadic Parkinson's disease (PD). In fact, rotenone, a complex I inhibitor, has been used for establishing PD models both in vitro and in vivo.

To date, there have been extremely limited options for treating mitochondrial dysfunction. For example, efforts to treat Parkinson Disease have been largely unsuccessful. Currently there is no specific treatment of mitochondrial dysfunction after myocardial infarction or in heart failure. Animal studies using lentivirus to deliver the NDI1 gene have shown promise, but technical challenges of gene therapy have limited its success.

### SUMMARY

The invention provides methods and compositions for treating, ameliorating or preventing diseases or conditions caused by or aggravated by lost and/or impaired mitochondrial Complex I function, including treating, ameliorating or preventing an ischemia and/or reperfusion injury, Parkinson's disease, myopathic diseases, cardiolipin deficiency, neurodegenerative diseases, aging, diabetes, obesity, sepsis and other conditions in which mitochondrial Complex I function is lost and/or impaired, by the administration of NDI1 chimeric (or fusion) proteins, including a chimeric NDI1-TAT protein, which can be completely or partially constructed as a recombinant protein and/or a peptidomimetic. In one aspect, methods and compositions of the invention are used in the treatment of mitochondrial dysfunction after myocardial infarction or in heart failure; compositions of the invention, e.g., TAT-NDI1, are effective for restoring normal mitochondrial function. In one aspect, methods and compositions of the invention are used in organ preservation for transplantation.

In addition to the exemplary NDI1-TAT chimeric (or fusion) protein, other NDI1 chimeric (or fusion) proteins of this invention, which can be used to practice the methods of this invention, include NDI1-biotin, NDI1-carnitine or NDI1-taurine conjugates. A chimeric polypeptide of this invention can be completely or partially constructed as a recombinant protein and/or as a peptidomimetic.

The invention provides chimeric isolated, synthetic or recombinant polypeptides comprising at least two domains or moieties, and having an NADH oxidoreductase activity (e.g., an isolated, synthetic or recombinant polypeptide having an NADH oxidoreductase activity and at least two domains or moieties), wherein the chimeric polypeptide comprises:

(a) (i) a first domain or moiety comprising an NDI1 polypeptide having an NADH oxidoreductase activity, and (ii) at least a second domain or moiety comprising or consisting of a polypeptide or a peptide (or, at least a second polypeptide or peptide domain or moiety);

(b) the chimeric polypeptide of (a)(i), wherein the NDI1 polypeptide the chimeric polypeptide of (a)(i), wherein the NDI1 polypeptide comprises or consists of a eukaryotic, a yeast, a Saccharomyces cerevisiae or a human NDI1 polypeptide;

(c) the chimeric polypeptide of (b), wherein the human NDI1 polypeptide comprises or consists of an amino acid sequence as set forth in SEQ ID NO:1;

(d) the chimeric polypeptide of any of (a) to (c), wherein the at least a second polypeptide domain or moiety comprises a TAT protein, a taurine, a biotin or a carnitine, or a cell or organelle targeting agent, or a mitochondrial targeting agent, or a carbohydrate-binding domain;

(e) the chimeric polypeptide of any of (a) to (d), wherein the at least a second polypeptide domain or moiety is located amino terminal, carboxy terminal, or amino terminal and carboxy terminal to the NDI1 polypeptide;

(f) the chimeric polypeptide of any of (a) to (e), further comprising a cationic moiety, or a cationic amino acid moiety, or a poly-arginine amino acid residue moiety, or equivalent;

(g) the chimeric polypeptide of (f), wherein the cationic moiety is located amino terminal, carboxy terminal, or amino terminal and carboxy terminal to the NDI1 polypeptide;

(h) a peptidomimetic of the chimeric polypeptide of any of (a) to (g); or

(i) the chimeric polypeptide of any of (a) to (g), or peptidomimetic of (h), further comprising, or modified by: acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation and/or arginylation;

(j) the chimeric polypeptide of any of (a) to (g), or peptidomimetic of (h), wherein the first domain or moiety is joined to the at least second domain or moiety by a chemical linking agent.

In one aspect, the chimeric proteins of this invention comprise fragments or altered or truncated forms of NDI1 protein, or equivalent. In other aspects, chimeric NDI1 proteins of the invention are joined or fused to other moieties such as cell targeting domains, organelle targeting domains, e.g., mitochondrial targeting domains, and the like.

The invention also provides pharmaceutical compositions comprising the chimeric NDI1 proteins of the invention, and methods of making and using them, including methods for treating, ameliorating or preventing an ischemia and/or reperfusion injury, Parkinson's disease, myopathic diseases, cardiolipin deficiency, neurodegenerative diseases, aging, diabetes, obesity, sepsis and other conditions in which mitochondrial Complex I function is lost and/or impaired. The invention also provides pharmaceutical compositions for the treatment of mitochondrial dysfunction after myocardial infarction or in heart failure. In one aspect, methods and compositions of the invention are used for organ preservation for transplantation. In one aspect, the chimeric polypeptides, pharmaceuticals and other compositions and methods of the invention are used for treating or ameliorating inflammation or injury where elevated levels of NADH/NADPH drive the production of reactive oxygen species by the respiratory burst oxidase or uncoupled nitric oxide synthase, and to lower the levels of the reduced forms of NADH/NADPH.

In one aspect, nucleic acids (e.g., chimeric isolated, synthetic or recombinant nucleic acids or polynucleotides) encoding chimeric NDI1 proteins of the invention. In one embodiment, these nucleic acids are transfected into a cell in vitro, ex vivo or in vivo for expression of the chimeric protein. In one aspect, nucleic acids encoding chimeric NDI1 proteins of the invention comprise DNA or RNA operably linked to a promoter. In one aspect, the nucleic acid comprises a plasmid DNA, a recombinant virus or phage, an expression cassette or a vector such as an expression vector. In one aspect, the cell is a bacterial cell, a fungal cell, a plant cell, a yeast cell, an insect cell, a mammalian cell, e.g., a human cell.

The invention provides methods for transfecting a cell with a nucleic acid of the invention (encoding a chimeric NDI1 protein of the invention) comprising the following steps: (a) providing a nucleic acid-comprising composition of the invention; (b) contacting the cell with the composition of step (a) under conditions wherein the composition is internalized into the cell. In one aspect, the transfecting is an in vivo transfection or an in vitro transfection.

In one aspect, a chimeric NDI1 protein of the invention comprises a plurality of cationic amino acid residues, or a cationic peptide moiety, e.g., comprises a plurality of arginines as a poly-arginine moiety, for increased intracellular penetration. See, e.g., Fuchs (2004) Biochemistry 43(9):2438-2444. In one aspect, the chimeric polypeptides and peptides of the invention are able to efficiently penetrate and enter cells in vivo because the cationic, e.g., poly-arginine, motif adds a positive charge.

In one aspect, a chimeric NDI1 protein of the invention has two or more different domains (one being NDI1) comprising recombinant, peptidomimetic and/or synthetic proteins wherein at least one domain (a first domain) is joined to another domain (a second, third, etc) domain or moiety by a chemical linking agent.

The invention provides compositions comprising (a) a first composition comprising a chimeric polypeptide of the invention; and a second composition; or (b) the composition of (a), wherein the second composition comprises a liquid, a lipid or a powder.

The invention provides liposomes comprising (a) the chimeric protein of the invention; or (b) the liposome of (a), wherein the liposome is formulated with a pharmaceutically acceptable excipient.

The invention provides pharmaceutical compositions comprising: the chimeric protein of the invention, the composition of the invention, or the liposome of the invention; and, a pharmaceutically acceptable excipient.

The invention provides inhalants or spray formulations comprising: the chimeric protein of the invention, or the composition of the invention, or the liposome of the invention, or the pharmaceutical composition of the invention; and, a pharmaceutically acceptable excipient.

The invention provides formulations (including a formulation for intrathecal, intraparenchymal or epidural administration, or parenteral administration into a perispinal space) comprising: the chimeric protein of the invention, or the composition of the invention, or the liposome of the invention, or the pharmaceutical composition of the invention; and, a pharmaceutically acceptable excipient; wherein the formulation can be a parenteral or enteral formulation. For example, the invention provides enteral formulations comprising: the chimeric protein of the invention, or the composition of the invention, or the liposome of the invention, or the pharmaceutical composition of the invention; and, a pharmaceutically acceptable excipient.

In one embodiment a composition (e.g., a formulation) of the invention is administered after an ischemic event in a heart or other organ, and/or with reperfusion of the heart or other organ.

The invention provides methods for treating, ameliorating or preventing the disease or condition in which mitochondrial Complex I function is lost and/or impaired, in an individual in need thereof, comprising:

(A) (a) providing the chimeric protein of the invention, the composition of the invention, the liposome of the invention, the pharmaceutical composition of the invention, the inhalant or spray formulation of the invention, the parenteral formulation of the invention, or the enteral formulation of the invention; and (b) administering an effective amount of (a) to the individual, thereby preventing, ameliorating or treating the disease or condition in which mitochondrial Complex I function is lost and/or impaired; or,

(B) the method of (A), wherein the disease or condition in which mitochondrial Complex I function is lost and/or impaired is an ischemia and/or reperfusion injury, Parkinson's disease, myopathic diseases, cardiolipin deficiency, neurodegenerative diseases, aging, diabetes or obesity.

The invention also provides compositions and methods for the treatment of mitochondrial dysfunction after myocardial infarction or in heart or other organ failure. In one aspect, methods and compositions of the invention are used for organ (e.g., heart, liver, kidney) preservation for transplantation.

The invention also provides compositions and methods for the treatment of CNS diseases such as Parkinson's Disease or Alzheimer's Disease comprising administering a composition of the invention into the CNS, e.g., administering a composition of the invention intrathecally (into cerebrospinal fluid), intraparenchymally or epidurally, or parenterally into a perispinal space, for treatment of a CNS disease such as Parkinson's Disease or Alzheimer's Disease.

The invention provides isolated, synthetic or recombinant nucleic acids comprising or consisting of:

(a) a nucleic acid sequence encoding a chimeric polypeptide or peptide of the invention;

(b) the nucleic acid sequence of (a), and further comprising or consisting of nucleic acid sequence encoding a polypeptide antigen, label or tag;

(c) the nucleic acid sequence of (b), wherein the polypeptide antigen, label or tag comprises or consists of a fluorescent or a detectable protein, or an enzyme, or an enzyme that generates a detectable agent or moiety.

The invention provides vectors, cloning or expression vectors, expression cassettes, plasmids, phages, or recombinant viruses comprising the isolated or recombinant nucleic acid of the invention (which encodes a chimeric protein of the invention).

The invention provides host cells comprising (a) the vector, cloning or expression vector, expression cassette, plasmid, phage, or recombinant virus of the invention (which comprise nucleic acid encoding a chimeric protein of the invention), or a recombinant nucleic acid encoding the polypeptide of the invention; or a nucleic acid of the invention; or (b) the host cell of (a), wherein the cell is a bacterial cell, a mammalian cell, a fungal cell, an insect cell, a yeast cell or a plant cell.

The invention provides non-human transgenic animals comprising (a) the vector, cloning or expression vector, expression cassette, plasmid, phage, or recombinant virus of the invention, or a recombinant nucleic acid encoding the polypeptide of the invention; or a nucleic acid of the invention; or (b) the non-human transgenic animal of (a), wherein the animal is a mouse or a rat.

The invention provides methods for transfecting a cell with a nucleic acid comprising: (a) providing a nucleic acid encoding a chimeric polypeptide of the invention, or a nucleic acid of the invention; and, (b) contacting the cell with the nucleic acid of (a) under conditions wherein the nucleic acid is internalized into the cell.

The invention provides pharmaceutical compositions comprising a chimeric polypeptide of the invention, or a nucleic acid of the invention, or a vector, cloning or expression vector, expression cassette, plasmid, phage, or recombinant virus of the invention.

The invention provides inhalants or spray formulations comprising the pharmaceutical composition of the invention; and, a pharmaceutically acceptable excipient. The invention provides parenteral formulations comprising the pharmaceutical composition of the invention; and, a pharmaceutically acceptable excipient. The invention provides enteral formulations comprising the pharmaceutical composition of the invention; and, a pharmaceutically acceptable excipient.

The invention provides methods an inhaler, nebulizer or atomizer comprising pharmaceutical composition of the invention. The invention provides uses of the chimeric protein of the invention, the composition of the invention, the liposome of the invention, or the inhalant or spray formulation of the invention to make a pharmaceutical composition.

The invention provides uses chimeric polypeptides of the invention to make a pharmaceutical composition. In one aspect, the pharmaceutical composition is made to treat, prevent or ameliorate an ischemia and/or reperfusion injury, Parkinson's disease, Alzheimer's Disease, myopathic diseases, cardiolipin deficiency, neurodegenerative diseases, aging, diabetes, obesity, sepsis and other conditions in which mitochondrial Complex I function is lost and/or impaired. The invention also provides methods for the treatment of mitochondrial dysfunction after myocardial infarction or in heart failure. In one aspect, methods and compositions of the invention are used for (a) organ preservation for transplantation or storage, or (b) skin, kidney, liver or heart storage and/or preservation for transplantation or storage.

The invention provides use of the chimeric proteins of the invention, or the peptidomimetics of the invention, or nucleic acids of the invention, to make a pharmaceutical composition for treating or ameliorating inflammation or injury where elevated levels of NADH/NADPH drive the production of reactive oxygen species by the respiratory burst oxidase or uncoupled nitric oxide synthase, and to lower the levels of the reduced forms of NADH/NADPH.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates data from studies where cardiomyocytes and neonatal cardiomyocytes were transiently transfected with either empty pHook-encoding or ndi1-encoding plasmid, and after 48 hr sI/R was performed with 24 hr reperfusion, as discussed in detail in Example 1, below.

FIG. 2 graphically illustrates data demonstrating expression of an Ndi1 polypeptide by transient transfection protects against sI/R by attenuating reactive oxygen species (ROS) and by preserving ATP, as discussed in detail in Example 3, below. Ndi1 was transiently transfected into HL-1 myocytes subjected to sI/R, as discussed in detail in Example 3, below: ATP levels and NAD⁺/NADH ratios were both increased by Ndi1 expression following sI/R; representative results are shown in FIGS. 2A, B, and C:

FIG. 2A graphically illustrating superoxide production as a function of average RFU in untransfected, pHOOK™ transfected, NDI1 transfected, untransfected with IR, pHOOK™ transfected with IR, and NDI1 transfected with IR samples, as indicated in the drawing; FIG. 2B graphically illustrating ATP production as a function of relative luminescent units (RLUs) in untreated (“UNT”), NDI1 transfected with IR and pHOOK™ transfected with IR sample, as indicated in the drawing; FIG. 2C graphically illustrating (NAD+/NADH) levels in the samples: untreated (“UNT”), pHOOK™ transfected with I/R, NDI1 transfected with I/R, and NDI1 transfected with I/R with the Ndi1 inhibitor flavone also added, as indicated in the drawing.

FIG. 3 schematically illustrates an exemplary plasmid of the invention, the so-called “pTAT-ndi1-HA”, which was used to express recombinant the exemplary Tat-NDI1 chimeric polypeptide of this invention in bacteria. The purified recombinant protein was then applied to cardiomyocytes; this protein protecting the cells from simulated ischemia/reperfusion, as discussed in detail in Example 3, below.

FIG. 4, lower panel, graphically illustrates data demonstrating that delivery of the exemplary chimeric polypeptide of the invention TAT-NDI1 to cells protects them against simulated ischemia/reperfusion (sI/R); FIG. 4, upper panel, illustrates the results of immunostaining with anti-NDI1 antibody (and a cytochrome c antibody); this staining reveals a mitochondrial distribution of TAT-NDI1 that co-localizes with cytochrome c, as discussed in detail in Example 3, below.

FIGS. 5A and 5B illustrate immunostains showing that Tat-Ndi1 is taken up into cardiomyocytes after perfusion into the isolated perfused heart. First (left) panel is control heart (no Tat-Ndi1 perfusion), stained with anti-Ndi1 antibody and FITC-conjugated secondary antibody. Second (middle) panel is heart that was perfused with Tat-Ndi1 and immunostained with anti-Ndi1 antibody (and FITC-conjugated secondary antibody). Third (right) panel is heart perfused with Tat-Ndi1 and stained with anti-HA antibody (and FITC-conjugated secondary antibody) (HA is an epitope that is contained in the Tat-Ndi1 recombinant protein). FIG. 5B left panel graphically illustrates immunostaining of an exemplary Tat-NDI1 chimeric polypeptide of the invention internalized into a cardiomyocte and stained with an anti-NDI1 antibody; FIG. 5B middle panel is an illustration of immunostaining of a cytochrome c with an anti-cytochrome c antibody; and FIG. 5B, right panel is a merged image of both the anti-NDI1 antibody and the anti-cytochrome c antibody immunostaining images

FIG. 6 lower right panel graphically illustrates data showing the infarct size (calculation of necrotic area as a percentage of total area) with (“NDI1”) and without (untreated, or “UNT”) administration of the exemplary chimeric TAT-NDI1 polypeptide of the invention; FIG. 6 lower left panel illustrates TTC staining of necrotic areas with (“NDI1”) and without (untreated, or “UNT”) administration of the exemplary chimeric TAT-NDI1; and FIG. 6 upper panel graphically illustrates the protocol and timing of this study, as discussed in detail in Example 3, below.

FIG. 7 illustrates data from a study where isolated rat hearts were perfused in Langendorff mode and Tat-Ndi1 or vehicle (control) was introduced into the perfusate, as discussed in detail in Example 3, below. After 30 min global no-flow ischemia and 30 min reperfusion, hearts were sliced and stained for superoxide production with dihydroethidium (FIG. 7A), or snap-frozen and processed to measure ATP content (FIG. 7B). This shows that Tat-Ndi1 preserves mitochondrial integrity reflected by diminished ROS production and increased ATP production.

FIG. 8 graphically illustrates spectrophotometry data from a study where isolated rat hearts were perfused with or without Tat-Ndi1, then mitochondria were isolated by polytron homogenization and differential sedimentation and subjected to calcium-induced swelling, as discussed in detail in Example 3, below. This shows that Tat-Ndi1 increases resistance to opening of the mitochondrial permeability transition pore.

FIGS. 9 and 10 illustrate and summarize data demonstrating Ndi1 as a therapeutic/prophylactic agent; isolated rat heart tissue sections are illustrated in FIG. 9; FIG. 10A illustrates a bar graph of data showing the reduction in infarct size (n=2 so far, but more in progress); FIG. 10B illustrates the protocol for this study, where the arrow indicates the time of administration of Tat-Ndi1 (at reperfusion), as discussed in detail in Example 3, below.

FIG. 11 graphically illustrates data demonstrating that administration of a yeast Ndi1 polypeptide is cytoprotective in cardiomyocytes, as discussed in detail in Example 3, below.

Like reference symbols in the various drawings indicate like elements.

### DETAILED DESCRIPTION

The invention provides methods and compositions for treating, ameliorating or preventing diseases or conditions caused by or aggravated by lost and/or impaired mitochondrial Complex I function, including treating, ameliorating or preventing an ischemia and/or reperfusion injury, Parkinson's disease, myopathic diseases, cardiolipin deficiency, neurodegenerative diseases, aging, diabetes, obesity, and other conditions in which mitochondrial Complex I function is lost and/or impaired, by the administration of chimeric proteins (including peptidomimetics) of this invention. The invention also provides methods for the treatment of mitochondrial dysfunction after myocardial infarction or in heart failure. In one aspect, methods and compositions of the invention are used for organ preservation for transplantation.

The invention provides compositions comprising chimeric NDI1 proteins as therapeutic proteins, including TAT-NDI1, taurine-NDI1, biotin-NDI1, carnitine-NDI1 and the like.

The chimeric proteins of the invention can be entirely or partly recombinant proteins, which can be expressed in any cell type, or in vitro. For example, the partially or completely recombinant chimeric proteins of the invention can be expressed in yeast, plant, bacteria, insect, fungal and/or mammalian cells, as a recombinant protein. In one aspect, they are purified and formulated for administration to animals, including, humans, to prevent, ameliorate and/or treat conditions in which it is desirable to replace or restore the function of mitochondrial electron transfer Complex I.

While the invention is not limited by any particular mechanism of action, in one aspect recombinant chimeric proteins of the invention are taken up by cells, e.g., taken up by cells ex vivo or in vivo, and enter the mitochondria where they function as an NADH oxidoreductase. The invention provides for the first time an effective mitochondrial delivery system for NDI1; the invention provides an NDI1-comprising chimeric recombinant protein that can be effectively delivered to mitochondria and therapeutically replace the function of a damaged or missing component of the electron transfer chain. Thus, in alternative aspects of the invention, the chimeric compositions and methods of the invention can have prophylactic and/or therapeutic applications in treatment of, e.g., ischemia/reperfusion injury, Parkinson Disease, various myopathic diseases, cardiolipin deficiency, neurodegenerative diseases, aging, diabetes, obesity, and other conditions in which mitochondrial Complex I function is impaired. The invention also provides methods for the treatment of mitochondrial dysfunction after myocardial infarction or in heart failure. In one aspect, methods and compositions of the invention are used for organ preservation for transplantation.

The chimeric compositions of the invention can comprise modified protein transduction domains and sequence variations or modifications of NDI1, as well as conjugated moieties including biotin, carnitine, taurine, and so forth.

In vivo or ex vivo delivery of oxidoreductase chimeric compositions of the invention can treat and/or ameliorate the impaired Complex I function which occurs after ischemia/reperfusion (IR); and because I/R is a potent source of reactive oxygen species (ROS), in vivo delivery of chimeric compositions of the invention can ameliorate or reduce the amount of and damage done by ROS. In neuronal models of Complex I deficiency, the yeast gene NDI1, can replace the function of Complex I with regard to NADH oxidoreductase activity (although not proton pumping), thereby restoring mitochondrial function, decreasing ROS production, and preserving cell viability. Accordingly, in vivo delivery of Ndi1-comprising chimeric compositions of the invention to the heart is cardioprotective and reduces post-ischemic tissue damage.

While the invention is not limited by any particular mechanism of action, in one aspect, the Ndi1-comprising oxidoreductase chimeric compositions of the invention are targeted to the mitochondrial inner membrane, resulting in cardioprotection and reduction of post-ischemic tissue damage.

### Generating and Manipulating Nucleic Acids and Polypeptides

The invention provides nucleic acids encoding chimeric NDI1 proteins of the invention, including partially or completely recombinant TAT-NDI1, taurine-NDI1, biotin-NDI1, carnitine-NDI1 and the like. The invention can be practiced in conjunction with any method or protocol or device known in the art, which are well described in the scientific and patent literature.

The invention provides “nucleic acids” or “nucleic acid sequences” encoding chimeric NDI1 proteins of the invention, including oligonucleotides, nucleotides, polynucleotides, or any fragments of these, including DNA or RNA (e.g., mRNA, rRNA, tRNA) of genomic or synthetic origin, which may be single-stranded or double-stranded and may represent a sense or antisense strand, to peptide nucleic acid (PNA), or to any

DNA-like or RNA-like material, natural or synthetic in origin, including, e.g., iRNA, ribonucleoproteins (e.g., iRNPs). The invention provides for use of ITK-inhibitory nucleic acids, i.e., oligonucleotides, containing known analogues of natural nucleotides, naturally occurring nucleic acids, synthetic nucleic acids, and recombinant nucleic acids.

The invention also encompasses use of nucleic-acid-like structures with synthetic backbones that encode chimeric NDI1 proteins of the invention, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. The invention provides for use of nucleic acids containing known analogues of natural nucleotides. The invention provides for use of mixed oligonucleotides comprising an RNA portion bearing 2′-O-alkyl substituents conjugated to a DNA portion via a phosphodiester linkage, see, e.g., U.S. Pat. No. 5,013,830. The invention provides for use of nucleic-acid-like structures with synthetic backbones to encode chimeric NDI1 proteins of the invention. DNA backbone analogues provided by (used when practicing) the invention include phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). The invention provides for use of PNAs containing non-ionic backbones, such as N-(2-aminoethyl)glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones that can be used when practicing this invention include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The invention provides for use of nucleic acids that encode all or part of the chimeric NDI1 proteins of the invention, including genes, polynucleotides, DNA, RNA, cDNA, mRNA, oligonucleotide primers, probes and amplification products.

The invention provides for use of chimeric NDI1 proteins comprising “amino acids” or “amino acid sequences” including an oligopeptide, peptide, polypeptide, or protein sequence, or to a fragment, portion, or subunit of any of these, and to naturally occurring or synthetic molecules. In one aspect, the invention provides for use of chimeric NDI1 proteins comprising “polypeptides” and “proteins” joined to each other by peptide bonds or modified peptide bonds, i.e., peptide isosteres, and may contain modified amino acids other than the 20 gene-encoded amino acids. The invention provides for use of chimeric NDI1 “polypeptides” including peptides and polypeptide fragments, motifs and the like. The chimeric NDI1 proteins of the invention also include glycosylated polypeptides. The invention provides for use of chimeric NDI1 polypeptides of the invention comprising partially or completely peptides and polypeptides including all “mimetic” and “peptidomimetic” forms.

The nucleic acids used to practice this invention, whether RNA, cDNA, genomic DNA, vectors, viruses or hybrids thereof, may be isolated from a variety of sources, genetically engineered, amplified, and/or expressed/generated recombinantly (recombinant polypeptides can be modified or immobilized to arrays in accordance with the invention). Any recombinant expression system can be used, including bacterial, mammalian, yeast, insect or plant cell expression systems.

In one aspect, the term “recombinant” includes nucleic acids adjacent to a “backbone” nucleic acid to which it is not adjacent in its natural environment. “Synthetic” polypeptides or protein are those prepared by chemical synthesis, as described in further detail, below.

Alternatively, nucleic acids of the invention, or nucleic acids used to practice the invention, can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with a primer sequence.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The nucleic acids used to practice this invention, whether RNA, iRNA, siRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Another useful means of obtaining and manipulating nucleic acids used to practice this invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In practicing the invention, nucleic acids of the invention or modified nucleic acids of the invention, can be reproduced by amplification. Amplification can also be used to clone or modify the nucleic acids of the invention. Thus, the invention provides amplification primer sequence pairs for amplifying nucleic acids of the invention. One of skill in the art can design amplification primer sequence pairs for any part of or the full length of these sequences.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990)Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; and Sooknanan (1995) Biotechnology 13:563-564.

### Chimeric NDI1 Polypeptides

The invention provides for use of chimeric NDI1 polypeptides isolated from natural sources, be synthetic, or be recombinantly generated polypeptides. Peptides and proteins can be recombinantly expressed in vitro or in vivo. The chimeric peptides and polypeptides of the invention can be made and isolated using any method known in the art. Chimeric polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The invention provides for use of chimeric NDI1 polypeptides that are glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The invention provides for use of chimeric NDI1 polypeptides in any “mimetic” and/or “peptidomimetic” form. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic (e.g., use of a mimetic) is within the scope of the invention, i.e., that its structure and/or function is not substantially altered; e.g., the chimeric polypeptide of the invention retains NADH oxidoreductase activity.

The invention provides for use of chimeric NDI1 polypeptide mimetic compositions comprising any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

The invention provides for use of chimeric NDI1 polypeptides characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

The invention provides for use of chimeric NDI1 polypeptides comprising mimetics of acidic amino acids generated by substitution by, e.g., non-carboxylate amino acids while maintaining a negative charge; (phosphono)alanine; sulfated threonine.

Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

The invention provides chimeric NDI1 polypeptides as described herein, further altered by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

The invention provides chimeric NDI1 polypeptides made by solid-phase chemical peptide synthesis methods. For example, assembly of a polypeptides or peptides of the invention can be carried out on a solid support using an Applied Biosystems, Inc. Model 431A™ automated peptide synthesizer. Such equipment provides ready access to the polypeptides or peptides of the invention, either by direct synthesis or by synthesis of a series of fragments that can be coupled using other known techniques.

The invention provides chimeric NDI1 polypeptides lacking a signal peptide or comprising a heterologous signal peptide.

### Pharmaceutical Compositions

The invention provides pharmaceutical compositions comprising an chimeric NDI1 polypeptide of the invention and a pharmaceutically acceptable excipient. The invention provides for uses of a chimeric NDI1 polypeptide of the invention to make a pharmaceutical composition. The invention provides parenteral formulations comprising a chimeric NDI1 polypeptide of the invention. The invention provides enteral formulations comprising a chimeric NDI1 polypeptide of the invention. The invention provides methods for treating, ameliorating and/or preventing an ischemia and/or reperfusion injury, Parkinson's disease, myopathic diseases, cardiolipin deficiency, neurodegenerative diseases, aging, diabetes, obesity, and other conditions in which mitochondrial Complex I function is lost and/or impaired, comprising providing a pharmaceutical composition comprising a chimeric NDI1 polypeptide of the invention; and administering an effective amount of the pharmaceutical composition to a subject in need thereof. The invention also provides methods for the treatment of mitochondrial dysfunction after myocardial infarction or in heart failure. In one aspect, methods and compositions of the invention are used for organ preservation for transplantation.

The pharmaceutical compositions used in the methods of the invention can be administered by any means known in the art, e.g., intrathecally, intraparenchymally or epidurally, perispinally, parenterally, topically, orally, or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Pharmaceutical formulations of the invention can be prepared according to any method known to the art for the manufacture of pharmaceuticals. Such drugs can contain sweetening agents, flavoring agents, coloring agents and preserving agents. A formulation of the invention can be admixtured with nontoxic pharmaceutically acceptable excipients which are suitable for manufacture. Formulations of the invention may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc, or formulated for inhalers, nebulizers, which are devices used to administer medication to people in forms of a liquid mist to the airways, or atomizers. A vaporized medicine can be inhaled through a tube-like mouthpiece, e.g., an inhaler, nebulizer or atomizer; this can have a benefit of allowing surrounding air to mix with the formulation, decreasing the unpleasantness of the vapor, if any.

For example, in one embodiment compositions of the invention can be delivered using a device comprising a nasal actuator with a asymmetric orifice opening that produces bimodal particle size distribution, e.g., delivered using a formulation in the form of a powder packaged under pressure which is released upon activation of an appropriate valve system; as described e.g., in U.S. Pat App Pub No. 20080029084. The compositions of the invention can be formulated as particles in a nebulized solution or powder that lodge along an upper and/or lower or deep respiratory tract. The compositions of the invention can be formulated as dry powders made by spray drying, e.g., with dual nozzles, or spray freeze drying with dual nozzles, or e.g., using a partially friable spray freeze dried powder with a dual particle size distribution, or e.g., by blending of milled freeze-dried or milled powders of two different particle sizes; see e.g., U.S. Pat App Pub No. 20080029084.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a chimeric polypeptide or peptidomimetic of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration of hydrophobic active agents of the invention. Oil-based suspensions can be formulated by suspending an active agent (e.g., a chimeric composition of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In the methods of the invention, the pharmaceutical compounds can also be administered by intrathecal, intraparenchymal, epidural, perispinal, intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In the methods of the invention, the pharmaceutical compounds can be delivered by transdermally, by a topical route, formulated as applicator sticks, solutions, suspensions, emulsions, gels, creams, ointments, pastes, jellies, paints, powders, and aerosols.

In the methods of the invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In the methods of the invention, the pharmaceutical compounds can be parenterally administered, such as by intravenous (IV) administration or administration into a body cavity or lumen of an organ, including intrathecally into the cerebrospinal fluid, intraparenchymally or epidurally, or by parenteral administration into a perispinal space. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

Liposomes

The compositions and formulations of the invention can be delivered by the use of liposomes. In one aspect, liposome of the invention are designed with surfaces carrying ligands specific for target cells, or ligands preferentially directed to a specific organ, to focus the delivery of the active agent of this invention into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

For example, in one embodiment, compositions and formulations of the invention are delivered by the use of liposomes having rigid lipids having head groups and hydrophobic tails, e.g., as using a polyethylene glycol (PEG)-linked lipid having a side chain matching at least a portion the lipid, as described e.g., in US Pat App Pub No. 20080089928. In another embodiment, compositions and formulations of the invention are delivered by the use of amphoteric liposomes comprising a mixture of lipids, e.g., a mixture comprising a cationic amphiphile, an anionic amphiphile and/or neutral amphiphiles, as described e.g., in U.S. Pat. App. Pub. Nos. 20080088046 or 20080031937. Amphoteric liposomes of the invention can comprise an active ingredient and at least one amphipathic cationic lipid, at least one amphipathic anionic lipid, and at least one neutral lipid, e.g., as described in U.S. Pat. No. 7,371,404.

In another embodiment, compositions and formulations of the invention are delivered by the use of liposomes comprising a polyalkylene glycol moiety bonded through a thioether group and an antibody also bonded through a thioether group to the liposome, as described e.g., in U.S. Pat. App. Pub. No. 20080014255. In another embodiment, compositions and formulations of the invention are delivered by the use of liposomes comprising glycerides, glycerophospholipids, glycerophosphinolipids, glycerophosphonolipids, sulfolipids, sphingolipids, phospholipids, isoprenolides, steroids, stearines, sterols and/or carbohydrate containing lipids, as described e.g., in U.S. Pat. App. Pub. No. 20070148220.

In one embodiment, compositions and formulations of the invention are delivered by the use of liquid-crystalline multi-molecular aggregates comprising a plurality of amphiphilic molecules dispersed in an aqueous solution, e.g., as described in U.S. Pat. No. 7,368,129.

In one embodiment, compositions and formulations of the invention are delivered to the respiratory tract of an individual via inhalation, e.g., using a nebulized liposomal aerosol, e.g., comprising a dilauroylphosphatidylcholine liposome, e.g., as described in U.S. Pat. No. 7,348,025.

In one embodiment, liposomes or other carrier vehicles for blood-brain barrier antigen compositions and formulations of the invention are delivered in a vehicle that specifically targets the blood brain barrier, e.g., by incorporating an antibody that specifically binds to a blood brain barrier molecule (antigen), e.g., the antibody FC5 or FC44, e.g., as described in U.S. Pat. App. Pub. No. 20090047300, or any antibody or receptor-binding molecules that specifically binds to a receptor that undergo transcytosis across the blood-brain barrier.

CNS Delivery

In one embodiment, c are delivered into the CNS, e.g., into the cerebrospinal fluid, by intrathecal administration, e.g., as described in U.S. Pat. No. 7,226,430; or parenterally into the perispinal space, e.g., as described in U.S. Pat. No. 7,214,658, or U.S. Pat. App. Pub. No. 20090130019; or intraparenchymally or epidurally. In one embodiment, compositions and formulations of the invention are delivered using retrograde venous perfusion to deliver compositions and formulations of the invention to the brain, eye, retina, auditory apparatus or cranial nerves e.g., as described in U.S. Pat. App. Pub. No. 20090130019.

In one embodiment, perispinal administration involves anatomically localized delivery performed so as to place a composition or formulation of the invention directly in the vicinity of the spine at the time of initial administration, e.g., into the “interspinous space”, including for example the subcutaneous and deeper areas, which is between two adjacent spinous processes but is external to the ligamentum flavum, which delimits the epidural space. In alternative embodiments, perispinal administration includes parenteral; subcutaneous; intramuscular; interspinous and/or epidural administration. Percutaneous injection can be carried through the skin in the midline of the neck or back, directly overlying the spine, to deliver a composition or formulation of the invention into the subcutaneous or deeper portion of the interspinous space; or, by percutaneous epidural injection to deliver directly into the epidural space.

In one embodiment, administration is by an indwelling epidural catheter for delivery into an epidural space; or, administration via an indwelling interspinous catheter into an interspinous space, e.g., a midline interspinous administration. Placement of an indwelling catheter to deliver a composition or formulation of the invention can be in the epidural space; in the interspinous space; or within the subarachnoid space; or by direct intrathecal administration.

In one embodiment, administration is by an intrathecally-implantable depots, e.g., having a biodegradable core for extended release of a composition or formulation of the invention into the intrathecal space over at time period of time, e.g., over days or one or more months, e.g., as described in U.S. Pat. App. Pub. No. 20090123508. An intrathecally-implantable depot can be in the form of a liquid solution, powder, granules, pellets, tablets, capsules, and the like, and can use any pharmaceutically acceptable excipient.

Any method, protocol or apparatus can be used to effect intrathecal, intraparenchymal or epidural administration of a composition or formulation of the invention. For example, the therapy may be given using an Ommaya reservoir which is in common use for intrathecally administering drugs. For example, a ventricular tube can be inserted through a hole formed in the anterior horn, and it is connected to an Ommaya reservoir installed under the scalp. The reservoir can be subcutaneously punctured to intrathecally deliver the composition or formulation of the invention, which is injected into the reservoir.

Any device for intrathecal, intraparenchymal or epidural administration of therapeutic compositions to an individual can be used, see e.g., U.S. Pat. No. 6,217,552. Alternatively, a composition or formulation of the invention can be administered intrathecally, intraparenchymally or epidurally by a single injection, or continuous infusion. Dosages can be in the form of a single dose administration or multiple doses.

Therapeutically Effective Amount and Dose

The formulations of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). In the methods of the invention, a pharmaceutical composition is administered in an amount sufficient to treat (e.g., ameliorate) or prevent asthma. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat the treat (e.g., ameliorate) or prevent asthma and/or its symptoms. For example, an exemplary pharmaceutical formulation for oral administration of chimeric polypeptide of the invention is in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The compositions and formulations of the invention can further comprise other drugs or pharmaceuticals, e.g., compositions for treating asthma and related symptoms or conditions. The methods of the invention can further comprise co-administration with other drugs or pharmaceuticals, e.g., compositions for treating asthma and related symptoms or conditions. For example, the methods and/or compositions and formulations of the invention can be co-formulated with and/or co-administered with antibiotics (e.g., antibacterial or bacteriostatic peptides or proteins), e.g., those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Kits

The invention provides kits comprising a chimeric (fusion) polypeptide of the invention, a chimeric (fusion) polynucleotide of the invention, or a pharmaceutical composition of the invention, including instructions on practicing the methods of the invention, e.g., directions as to indications, dosages, patient populations, routes and methods of administration.

The invention will be further described with reference to the following examples; however, it is to be understood that the invention is not limited to such examples.

### EXAMPLES

### Example 1

### Demonstrating the Efficacy of Compositions of the Invention

The following example describes making and using exemplary oxidoreductase chimeric NDI1 protein compositions of the invention, and provides data demonstrating the efficacy of the methods and compositions of the invention for ameliorating ischemia/reperfusion injury to Complex I. While the invention is not limited by any particular mechanism of action, the invention provides methods and compositions of the invention for reducing Complex I is damage during ischemia/reperfusion.

While the invention is not limited by any particular mechanism of action, the chimeric NDI1 protein compositions of the invention can replace or supplement NADH quinone oxidoreductase function of Complex I. In one aspect, the chimeric NDI1 protein compositions of the invention can replace Complex I function in the post-ischemic heart and thereby improve cardiac function.

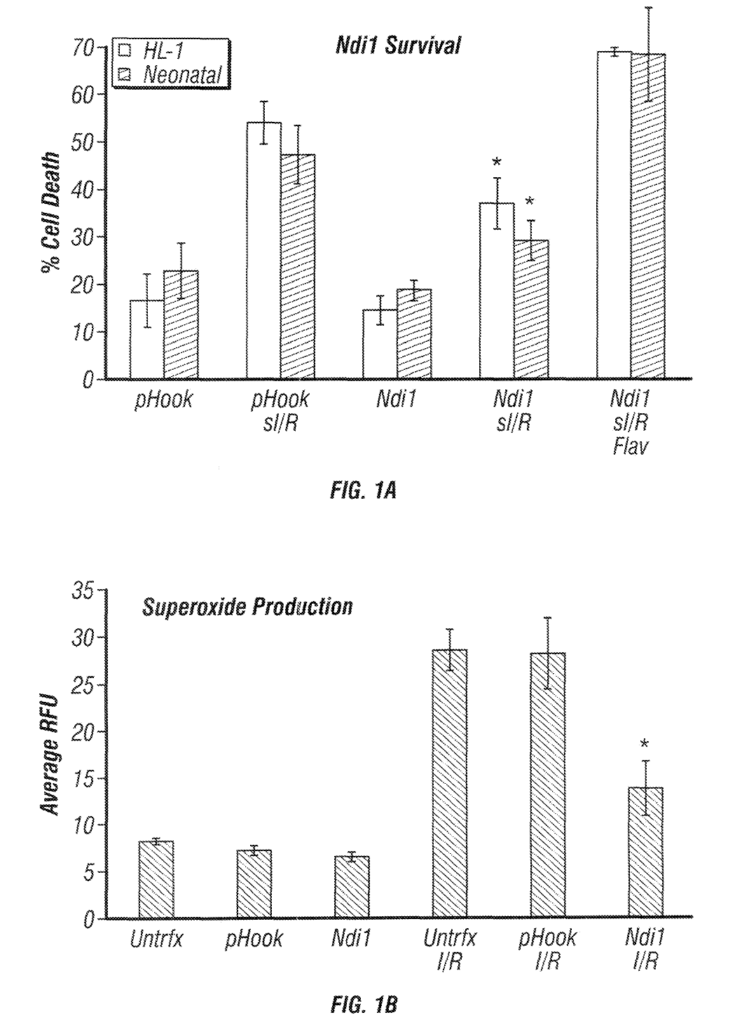

The NDI1 single polypeptide is not as hydrophobic as most mammalian complex I subunits, contains a single non-covalently bound FAD, and has no iron-sulfur or heme groups, making it an acceptable candidate for recombinant chimeric protein expression, e.g., as a Tat-mediated transduction. Studies indicated that after simulated ischemia/reperfusion (sI/R), neonatal myocytes and HL-1 cells produced elevated levels of ROS and subsequently died. We found that transient transfection of Ndi1 in HL-1 cells and neonatal rat cardiomyocytes could attenuate ROS production and preserve cell viability, demonstrating that Ndi1 was protective. This protection was dependent upon Ndi1 function, because flavone, a specific inhibitor of Ndi1, abolished protection; as illustrated in FIG. 1.

In FIG. 1, HL-1 cardiomyocytes and neonatal cardiomyocytes were transiently transfected with either empty pHook or ndi1 plasmid and after 48 hr sI/R was performed with 24 hr reperfusion. Apoptotic cells were detected by positive YoPro-1 staining and only transfected cells were scored. Flavone, a specific inhibitor of Ndi1, was administered before sI/R. Data expressed as % of total transfected cells and 200-300 cells per treatment were scored (n=3). Intracellular superoxide production after sI/R in neonatal cardiac myocytes was detected by DHE staining. DHE was imaged by fluorescence microscopy and quantified by fluorescence plate reader assay (excitation/emission 485 nm/580 nm) (n=6)*p<0.05.

NDI1-Comprising Compositions of this Invention can Substitute for Complex I and Confer Protection in I/R:

Viral gene delivery is impractical for the treatment of myocardial ischemia/reperfusion; accordingly, this invention provides Ndi1 chimeric polypeptides for therapeutic applications. Ndi1 is cloned into a standard construct as described in Gustafsson, et al. (2005) TAT-mediated protein transduction: delivering biologically active proteins to the heart. Methods Mol. Med. 112:81-90. In one aspect of the invention, Ndi1 is delivered to the matrix side of the inner mitochondrial membrane (IMM). Proteinase K susceptibility in the presence and absence of digitonin is used to map the submitochondrial localization of the fusion protein, as described in Yuan, et al. (2001) Differential processing of cytosolic and mitochondrial caspases. Mitochondrion 1:61-9. Ndi1 possesses the appropriate mitochondrial targeting sequence; however, the invention provides alternative embodiments for placing the second moiety of the chimeric polypeptide of the invention, including placement at the C-terminus or the N-terminus (an endogenous mitochondrial localization sequence is at the N-terminus). Additionally, a cleavable sequence can be inserted between the two domains, or moieties, of the chimeric polypeptide of this invention, e.g., as described by Albarran, et al. (2005) A TAT-streptavidin fusion protein directs uptake of biotinylated cargo into mammalian cells. Protein Engineering, Design and Selection 18:147-52. In this alternative embodiment, a recombinant protein is expressed and then biotinylated in vitro. The biotinylated recombinant protein, i.e., the biotin-Ndi1 chimeric protein of this invention, is then incubated with Tat-streptavidin which is expressed and purified as a separate recombinant protein. The advantage of this alternative embodiment is that after the protein complex enters the cell in an endosome, the biotin-tagged protein dissociates from streptavidin, an effect which is enhanced in the acidic endosomal compartment. This effect can be further facilitated by the simultaneous inclusion of the biotinylated pH-responsive polymer poly(propyl-acrylic acid), as described e.g., by Rinne, et al. (2007) Internalization of novel non-viral vector TAT-streptavidin into human cells. Bmc Biotechnology 7.

The following protocols can be used to validate successful delivery and efficacy of a chimeric protein of this invention, e.g., a Tat-Ndi1, whether made as a single fusion protein or as the biotinylated two-component complex:

Testing Tat-Ndi1 for Protection in HL-1 Cells.

We have shown that transient transfection of Ndi1 is protective against simulated I/R in HL-1 cells. If a chimeric protein, e.g., a Tat-Ndi1, is able to be delivered to the right compartment, it should also be protective. HL-1 cells can be treated with the chimeric protein to be tested, then subjected to sI/R. Protection can be monitored by measuring mitochondrial membrane potential (Rhodamine-123) and nuclear condensation (DAPI) after 5 hr reperfusion. If the Tat-streptavidin/biotin conjugate of the invention (e.g., biotin-Ndi1) approach is not successful, an alternative approach making a single fusion protein with the second domain (e.g., a TAT) located at the C-terminus, thus preserving exposure of the N-terminal mitochondrial targeting sequence; or alternatively, a TAT or other second domain can be on the N-terminus of Ndi1.

Documenting Subcellular Localization and Verifying Submitochondrial Localization

Once protection is demonstrated, cells can be treated with a chimeric protein of this invention, e.g., a Tat-Ndi1, for 60 min, then cell fractions (e.g., cytosol, nuclei, heavy membranes, light membranes) are isolated to evaluate the distribution of the chimeric protein (or simply Ndi1 if a cleavable sequence is placed between the Ndi1 first domain and the second domain, and the domains in fact are cleaved apart). The majority of the Ndi1, or chimeric protein (e.g., Tat-Ndi1) will associate with mitochondria. Detection of Ndi1 can be done by Western blot (e.g., antibody to Ndi1, also streptavidin-peroxidase will bind to the biotin).

Submitochondrial localization can be evaluated by proteinase K digestion in the presence/absence of digitonin as described by Yuan (2001), supra. Additional confirmation can be performed using thin sections of cell pellets prepared for electron microscopy and labeled with streptavidin-colloidal gold.

Evaluating Cardioprotection in Isolated Perfused Hearts Subjected to I/R

Langendorff heart perfusion studies can be used to evaluate cardioprotection: in one exemplary protocol, a chimeric protein of the invention is infused before global ischemia is induced (30 min) and reperfusion (up to 2 hr). Creatine kinase release is measured, infarct size is measured (e.g., by triphenyl tetrazolium staining), and hemodynamic function is measured to determine functionally the extent infusion of the tested chimeric protein of the invention is beneficial.

ROS production can also be measured using dihydroethidium staining, e.g., as described in Gustafs son (2002) TAT protein transduction into isolated perfused hearts: TAT-apoptosis repressor with caspase recruitment domain is cardioprotective. Circulation 106:735-9; or Hamacher-Brady et al. (2006) Response to myocardial ischemia/reperfusion injury involves Bnip3 and autophagy. Cell Death Differ. 14:146-57.

A chimeric protein of the invention, e.g., a Tat-Ndi1, also can be evaluated when delivered after ischemia in isolated perfused hearts—to evaluate a chimeric protein for its therapeutic efficacy. If a chimeric protein is protective, it also can be evaluated for how long a delay can be incurred between the ischemic event and administration to still confer any therapeutic benefit. For these studies, hemodynamic analysis is the most sensitive indicator.

In Vivo Models of I/R

We have established a surgical model of ischemia/reperfusion (I/R) in mice and rats: in one exemplary protocol, a chimeric protein of the invention, e.g., a Tat-Ndi1, is administered as an intraperitoneal (i.p.) injection 30-60 minutes before I/R. Uptake in the heart can be monitored by western blot (e.g., anti-Ndi1 and/or streptavidin-peroxidase), and a dose range can be established, and an optimal time of administration can be established, in preliminary studies before performing I/R surgeries. Once optimized doses and timing is established, regional ischemia is performed (30 min) and reperfusion is performed (up to 4 hrs), to determine infarct size and area at risk, e.g., as described in the rabbit studies in Granville, et al., (2004) Reduction of ischemia and reperfusion-induced myocardial damage by cytochrome P450 inhibitors. Proc. Natl. Acad. Sci. USA 101:1321-1326.

These studies can be repeated by administering a chimeric protein of the invention, e.g., a Tat-Ndi1, after ischemia if the studies in isolated perfused hearts indicate this is feasible.

Preventing MPTP Using Chimeric Polypeptides of this Invention

While the invention is not limited by any particular mechanism of action, given the predicted importance of Complex I in the mitochondrial permeability transition pore (MPTP), the NADH dehydrogenase activity of the Ndi1 component of a chimeric polypeptide of this invention, which replaces endogenous NADH dehydrogenase activity, can ameliorate the MPTP. Yeast mitochondria do not exhibit MPTP, suggesting that the vastly more complicated structure of mammalian Complex I is required. Accordingly, susceptibility of to MPTP in mitochondria from cells or tissues can be compared using a chimeric polypeptide of this invention, e.g., Tat-Ndi1, and a protein control.

### Materials and Methods

Cell culture. HL-1 cells, neonatal and adult cardiomyocytes are prepared and cultured as previously described in Karwatowska-Prokopczuk (1998) Circ. Res. 82:1139-1144; Brady (2007) FEBS Journal 274:3184-97; He (1999) Cell Death Differ. 6:987-991. Adenovirus and transfection reagents can include YFP-Bid-CFP (see e.g., Brady (2007) FEBS Journal, supra); Bax-mCherry; mutant Bax-mCherry; Cyp-D; catalytically inactive

Cyp-D, and Ndi1. Recombinant proteins can include GST-Bid, Tat-BH4, Tat-Ndi1, and Cyp-D. Live cell imaging will be performed as previously described e.g., in Karwatowska-Prokopczuk (1998) Circ. Res, supra.

Mitochondrial isolation from mouse and rat hearts. Hearts can be removed while still beating from mice anesthetized with Ketamine/Xylazine. Two mouse hearts are pooled and rapidly minced in ice cold MSE buffer (in mmol/L, mannitol 220, sucrose 70, EGTA 2, MOPS 5 [pH 7.4], and taurine 2 supplemented with 0.2% BSA). Heart tissue is homogenized in MSE with a polytron type tissue grinder at 11,000 RPM for 2.5 seconds followed by 2 quick strokes at 500 RPM with a loose fit Potter-Elvenhjem tissue grinder. The homogenate is centrifuged at 500 g twice for 5 minutes saving the supernatant. The pellet contains interfibrillar mitochondria which must be isolated using a brief trypsin digestion as described by Hoppel, see e.g., Lesnefsky (2001) Arch. Biochem. Biophys. JID—0372430 2001; 385:117-28; Palmer (1977) J. Biol. Chem. 252:8731-8739.

Subsarcolemmal mitochondria are sedimented from the supernatant at 3000 g twice, rinsing the pellet with MSE buffer. The final pellet is rinsed and resuspended in 50 ul Incubation medium (in mmol/L, mannitol 220, sucrose 70, EGTA 1, MOPS 5[pH 7.4], taurine 2, MgCl₂ 10, and KH₂PO₄ 5, supplemented with 0.2% BSA). Mitochondria are incubated for 15 minutes on wet ice and protein concentration is determined with BSA as a standard by a Bradford assay. All work is performed on wet ice at 0° C.

Mitochondrial swelling assay. Mitochondria are incubated in chambers of a 96-well plate in a fluorescence plate reader in mitochondrial respiration buffer supplemented with complex I substrate pyruvate 5 mM, malate 5 mM or complex II substrate succinate 5 mM with 2 mM ADP. Rotenone 2 μM and calcium 250 μM are added to mitochondria and swelling was monitored by following the decrease in absorbance at 520 nm.

Amplex Red assay for H₂O₂. Conditions were identical to the swelling assay except that Amplex Red Hydrogen Peroxide/Peroxide assay kit, (Molecular Probes) are used, and fluorescence is measured with excitation 560 nm and emission 590 nm.

Preparation of Mitoplasts and Submitochondrial Particles. Mitoplasts are Prepared by hypotonic swelling as described e.g., in Yuan (2003) Mitochondrion 2:237-244; Yuan (2001) Mitochondrion 1:61-69. Submitochondrial particles are prepared from mitoplasts by sonication and ultracentrifugation as described, e.g., in He (2001) Circulation Research 89:461-467.

Oxygen consumption measurements. Oxygen consumption is measured at 30° C. with a Clark type oxygen electrode, Instech, in 600 μl KCL respiration buffer (in mmol/L, KCL 140, EGTA 1, MOPS10 [pH 7.4], MgCl₂ 10, and KH₂PO₄ 5, supplemented with 0.2% BSA). Complex I activity is measured using 150 μg mitochondria with palmitoyl-L-carnitine, 40 μM, as a substrate and malate, 2.5 mM, as a counter ion. Complex II activity is measured using 150 μg mitochondria with succinate, 5 mM, as a substrate. Complex IV activity is measured using 100 μg mitochondria with TMPD, 0.4 mM/ascorbate 1 mM, as a substrate. For each complex the ADP stimulated respiration rate (state 3) is measured after the addition of 120 mM ADP, the ADP independent respiration rate, oligomycin-insensitive, (state 4) is measured after the addition of 2 μM oligomycin and the maximal respiration rate is measured following uncoupling the mitochondria with 2 μM FCCP. Rates are calculated as nA O₂/min/mg protein as the fraction sensitive to the inhibitors rotenone 2 mM for complex I, antimycin A 1 μM for complex II and KCN 1 mM for complex IV. As a measure of mitochondrial integrity, the respiratory control ratio state 3 divided by state 4 is calculated.

Complex I isolation. Complex I is recovered from 5 mg of mitochondrial fraction protein in 1 ml buffer A [50 mM Tris-HCl, pH 7.5, 1:100 Protease Inhibitor Cocktail Set I (Calbiochem), 1 mM PMSF, pH 7.5] containing 1% n-dodecylβ-D-maltoside, incubated for 30 min on ice, and centrifuged for 30 min at 21,000 g at 4° C. Complex I Capture Matrix (MitoSciences) is added and incubated overnight at 4° C., followed by 2 h incubation at room temperature. After being centrifuged 3 min at 3200 g, 4° C., the pellet is washed two times for 5 min with buffer A, and then resuspended in 400 of 1% SDS and incubated 10 min at room temperature. After centrifugation for 3 min at 3200 g, 4° C., protein A agarose is added to the supernatant, incubated 1 h at room temperature, and centrifuged as before, again saving the supernatant.

A biochemical method for Complex I isolation is based on solubilizing mitochondria with n-dodecyl-B-D-maltoside (Anatrace, Maumee, Ohio) and purification on a Q-Sepharose HP column (Amersham Biosciences) followed by ammonium sulfate precipitation and gel filtration on Sephacryl S-300 HR, see e.g., Carroll (2003) Mol. Cell. Proteomics 2:117-126.

Isolated perfused rat hearts. Langendorff-perfused rat hearts will be subjected to 30 min global ischemia and up to 2 hr reperfusion with Tat-mediated protein transduction as previously described e.g., in Gustafsson (2002) supra.

Tat-mediated protein transduction. Recombinant TAT-fusion protein expression and purification are performed as described e.g., in Gustafsson (2002) supra. In brief, a 500 mL LB ampicillin overnight culture of TAT-fusion protein is grown in the presence of 100 μmmol/L isopropylthiogalactoside (Sigma) at 37° C. with shaking. The bacterial pellet can be isolated by centrifugation, washed with PBS, resuspended in 10 mL buffer Z (8 mol/L urea, 100 mmol/L NaCl, and 20 mmol/L HEPES, pH 8.0), and sonicated on ice 3 times with 15-second pulses. The sonicate can be clarified by centrifugation at 20,000 g at 4° C. for 20 minutes. The clarified lysate can be equilibrated in 20 mmol/L imidazole and applied at room temperature to a pre-equilibrated 25-mL column packed with 5 mL Ni-NTA resin in buffer Z, including 20 mmol/L imidazole. The column can be allowed to proceed by gravity flow, and the flow-through was then reapplied. The column is washed with 50 mL of 20 mmol/L imidazole in buffer Z, and the fusion protein is eluted from the Ni-NTA column at concentrations of imidazole of 100 and 250 mmol/L in buffer Z; the 100- and 250-mmol/L fractions can be pooled and desalted into 1×PBS on PD-10 columns. The fusion proteins can be applied in 2.5-mL aliquots and eluted with 3.5 mL PBS supplemented with 0.5M NaCl and 10% glycerol. Chimeric proteins, e.g., TAT fusion proteins, can be stored at 4° C. and used within one week.

1. TAT-protein Transduction

1.1 TAT-protein Transduction into Langendorff Perfused Hearts
1. Excise the heart from anesthetized rat and quickly cannulate onto the Langendorff perfusion apparatus.
2. Perfuse the heart at a constant pressure of 60 mm Hg with Krebs-Ringer buffer (11.1 mM Glucose, 25 mM NaHCO₃, 2.5 mM CaCl₂, 4.7 mM KCl, 118.5 mM NaCl, 1.18 mM KH2PO4, 1.18 mM MgSO₄) and bubble the perfusate with a mixture of 95% O₂ and 5% CO₂ at 37° C.
3. Equilibrate the heart for 5 min in Krebs-Ringer buffer.
4. Add the TAT-ndi1 protein (50-100 nM) to the perfusion buffer and perfuse the heart for 15 min while re-circulating the buffer.
5. Subject the heart to global ischemia for 30 min by turning off the perfusion system.
6. After 30 min of ischemia, turn on the perfusion system to start reperfusion.
7. Reperfuse heart for 2 hours.
8. Non-transduced TAT protein is washed out during re-perfusion with Krebs-Ringer buffer.

2. Detection of TAT-Protein Transduction

Protein transduction and localization can be determined in two ways. The first method is by staining TAT-ndi1 with either the Ndi1 antibody or the HA-antibody and visualization of uptake by fluorescent microscopy. The other is by western blot analysis using an antibody specific for the TAT-fusion protein or the HA tag.

2.1 Detection of TAT-Protein by Immunohistochemistry

1. Embed heart in Tissue Tek OCT and freeze in liquid Nitrogen.

2. Cut cryosections at a thickness of 3 μm.

3. Rinse sections with 1×PBS for 5 min.

4. Add 50 μl Ndi1 polyclonal antibody diluted in PBS1:200 to slide

5. Incubate on rocker in humid chamber overnight at 4° C.

6. Wash in 1×PBS twice for 10 min.

7. Add 50 μL goat anti-rabbit Alexa Fluor 488 diluted 1:1000 in PBS.

8. Incubate in humid chamber 1 hr on rocker at room temperature.

9. Wash in 1×PBS twice for 10 min.

10. Stain the sections with 30 μg/ml Hoechst 33342 for 10 min to visualize nuclei.

11. Rinse in PBS.

12. Mount coverslip on slide.

13. Visualize by fluorescent microscopy.

2.2 Detection of TAT-Protein in Heart Tissue by Western analysis

1. Homogenize the TAT-protein perfused heart by Polytron in lysis buffer.

2. Clear the lysate by centrifugation at 20,000×g for 20 min at 4° C.

3. Separate proteins by SDS-PAGE and transfer to nitrocellulose membrane.

4. Probe for TAT-protein using either anti-Ndi1 or anti-HA antibody.

### Example 2

### Chimeric NDI1 Protein and Nucleic Acid Compositions of the Invention

The invention provides nucleic acids encoding chimeric NDI1 protein compositions of the invention. While the invention is not limited by any particular mechanism of action, the chimeric NDI1 proteins of the invention have an NADH oxidoreductase activity, and in alternative aspects are used in prophylactic and/or therapeutic applications in treatment, amelioration or prevention of, e.g., ischemia/reperfusion injury, Parkinson Disease, various myopathic diseases, cardiolipin deficiency, neurodegenerative diseases, aging, diabetes, obesity, sepsis and other conditions in which mitochondrial Complex I function is impaired. The invention also provides methods for the treatment of mitochondrial dysfunction after myocardial infarction or in heart failure. In one aspect, methods and compositions of the invention are used for organ preservation for transplantation.

The nucleic acids used to practice this invention include may be isolated from a variety of sources, genetically engineered, amplified, and/or expressed/generated recombinantly. The nucleic acids used to practice this invention can be expressed using any recombinant expression system, including bacterial, mammalian, yeast, insect or plant cell expression systems.

In one embodiment, NADH:ubiquinone oxidoreductase, or Ndi1p, is from a non-human source, e.g., a yeast, e.g., a Saccharomyces, such as a Saccharomyces cerevisiae, and any of these Ndi1p polypeptides, or enzymatically active variants and/or fragments thereof, can be used in a chimeric (fusion) protein of the invention, e.g., as the Saccharomyces cerevisiae Ndi1p having a sequence as set forth in SEQ ID NO:1:

| | (SEQ ID NO: 1) |
|---|---|
| 1 | mlsknlysnk rlltstntlv rfastrstgv ensgagptsf ktmkvidpqh sdkpnvlilg |
| 61 | sgwgaisflk hidtkkynvs iisprsyflf tpllpsapvg tvdeksiiep ivnfalkkkg |
| 121 | nvtyyeaeat sinpdrntvt ikslsaysql yqpenhlglh qaepaeikyd ylisavgaep |
| 181 | ntfgipgvtd yghflkeipn sleirrtfaa nlekanllpk gdperrrlls ivvvgggptg |
| 241 | veaagelqdy vhqdlrkflp alaeevqihl vealpivinm fekklssyaq shlentsikv |
| 301 | hlrtavakve ekqllaktkh edgkiteeti pygtliwatg nkarpvitdl fkkipeqnss |
| 361 | krglavndfl qvkgsnnifa igdnafaglp ptaqvahqea eylaknfdkm aqipnfqknl |
| 421 | ssrkdkidll feennfkpfk yndlgalayl gseraiatir sgkrtfytgg glmtfylwri |
| 481 | lylsmilsar srlkvffdwi klaffkrdff kgl |

See, e.g., Bowman (1997) Nature 387 (6632 Suppl):90-93; and NCBI GenBank ref no. NP_{—}013586.

### Example 3

### Chimeric NDI1 Protein of the Invention Effective in Treating I/R

The invention provides a therapeutic protein, called TAT-NDI1, which can be expressed in cells, e.g., bacteria, yeast, fungal cells, and the like, as a recombinant protein, purified, and which can then be administered to animals or humans to treat conditions in which it is desirable to replace or restore the function of mitochondrial electron transfer Complex I. Data presented in this example demonstrates the ability of the TAT-Ndi1 of this invention to transverse the cell membrane and correctly target to the mitochondria. Data presented in this example demonstrates the cardioprotective capacity of the Ndi1-comprising composition of this invention in ischemia reperfusion injury.

While the invention is not limited by any particular mechanism of action, the recombinant proteins of this invention are taken up by cells and enters the mitochondria where it functions as an NADH oxidoreductase. The recombinant proteins of this invention will have therapeutic value in treatment of ischemia/reperfusion injury, Parkinson Disease, various myopathic diseases, cardiolipin deficiency, neurodegenerative diseases, aging, diabetes, obesity, and other conditions in which mitochondrial Complex I function is impaired.

While the invention is not limited by any particular mechanism of action, the recombinant proteins of this invention are effective for treating mitochondrial dysfunction. This approach represents the first case where a recombinant protein can be delivered to mitochondria and replace the function of a damaged component of the electron transfer chain. Mammalian Complex I consists of 46 subunits and is extremely vulnerable to damage by proteolysis or oxidative stress. In yeast, however, the NADH oxidoreductase activity of Complex I is carried out by a single polypeptide, NDI1. Although this protein cannot perform the protein pumping activity of mammalian Complex I, it can function as an oxidoreductase and can transfer electrons to ubiquinone to initiate electron transport, thereby serving as a functional replacement for Complex I in mammalian cells. In one embodiment, Tat-NDI1 is synthesized in bacteria, yeast, fungal cells, and the like, as a recombinant protein, purified and used for therapeutic administration in humans.

In one embodiment, recombinant proteins of this invention are used to decrease or ameliorate the neurodegenerative process in Parkinson's Disease by decreasing or slowing the loss of dopaminergic neurons in the substantia nigra, where the earliest defect is impaired mitochondrial respiration due to a defect in Complex I. In one embodiment, recombinant proteins of this invention restore Complex I, and thus are an effective treatment for Parkinson's Disease. The administration of recombinant proteins of this invention, including TAT-NDI1, of this invention will circumvent many of the limitations of gene therapy because it is taken up readily by most (all) cells and tissues, and can cross the blood brain barrier.

In one embodiment, recombinant proteins of this invention are a specific treatment for mitochondrial dysfunction after myocardial infarction or in heart failure; TAT-NDI1 has the ability to restore mitochondrial function, and thus can be used to decrease or ameliorate damage in acute myocardial infarction, in organ preservation for transplantation, in heart failure, and in sepsis. In one embodiment, recombinant proteins of this invention, e.g., TAT-NDI1, are effective in settings of inflammation or other injury where elevated levels of NADH/NADPH drive the production of reactive oxygen species by the respiratory burst oxidase or uncoupled nitric oxide synthase, by lowering the levels of the reduced forms of NADH/NADPH.

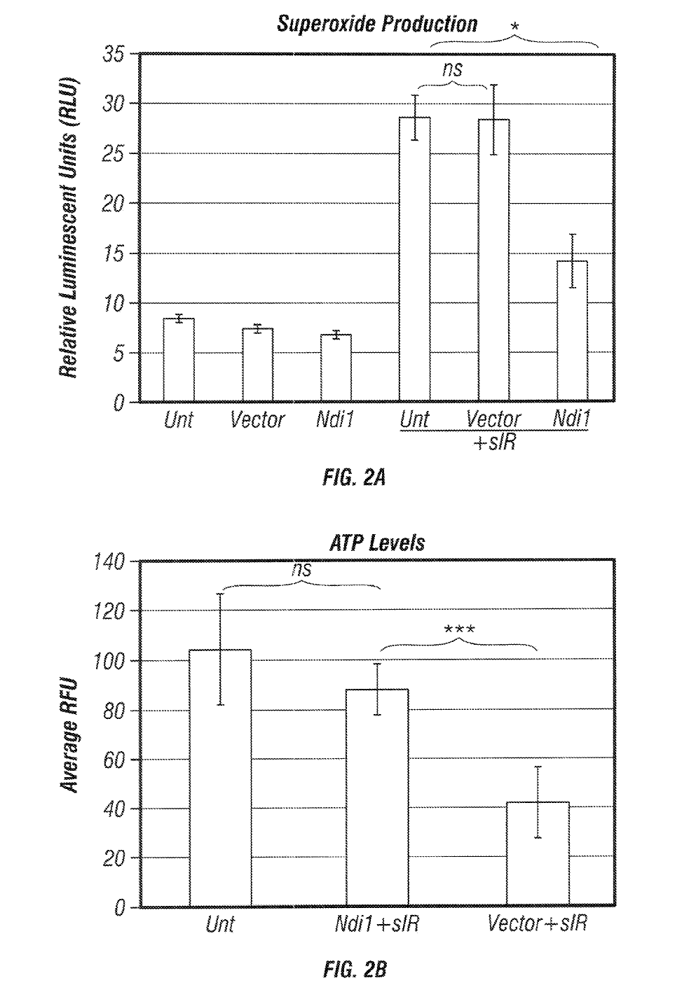

We have successfully cloned, purified, and expressed Tat-NDI1, and have shown that it can be added to HL-1 cardiomyocytes and can protect them from simulated ischemia/reperfusion. The plasmid used to express recombinant Tat-NDI1, called “pTAT-ndi1-HA”, this exemplary plasmid of the invention, pTAT-ndi1-HA, is schematically illustrated in FIG. 2, and uses a T7 promoter at positions 18 to 38; a 6× his tag at 110 to 127, TAT-PTD at 213 to 245, an HA tag at 269 to 294, and the ndi1 insert at 314 to 2101.

The sequence of the Tat-NDI1 is:

| | GTACCAGTTT CATCACATCA TCGAATTACA CGTTTACCCA |
|---|---|
| 351 | AGAAAAGAAA CTAAAAACCA CTATGCTATC GAAGAATTTG TATAGTAACA |
| 401 | AGAGGTTGCT CACCTCGACG AATACGCTAG TCAGATTCGC TTCCACCAGA |
| 451 | TCCACAGGGG TGGAAAACTC CGGAGCAGGT CCTACATCTT TTAAGACCAT |
| 501 | GAAAGTCATT GACCCTCAGC ACAGCGACAA ACCAAACGTG CTGATACTGG |
| 551 | GTTCGGGGTG GGGAGCTATT TCGTTTTTAA AGCACATTGA CACCAAGAAG |
| 601 | TACAACGTTT CCATCATCTC TCCTAGAAGC TATTTCTTAT TTACGCCTTT |
| 651 | GTTACCTTCT GCACCAGTTG GGACAGTAGA CGAAAAGTCA ATTATTGAGC |
| 701 | CCATCGTTAA TTTTGCTCTC AAGAAAAAGG GGAACGTTAC CTACTATGAG |
| 751 | GCAGAAGCCA CCTCTATCAA TCCCGACAGG AATACCGTTA CCATAAAATC |
| 801 | ATTATCTGCC GTTAGCCAGC TATACCAACC TGAAAACCAT CTAGGGCTGC |
| 851 | ATCAAGCAGA ACCTGCTGAA ATTAAGTACG ATTATTTAAT CAGTGCTGTA |
| 901 | GGTGCGGAAC CTAACACATT TGGTATTCCT GGGGTCACTG ATTACGGTCA |
| 951 | TTTCCTGAAG GAAATTCCCA ACTCTTTGGA AATAAGAAGA ACTTTTGCCG |
| 1001 | CCAATCTAGA GAAGGCTAAC TTATTGCCAA AGGGTGATCC CGAAAGAAGA |
| 1051 | AGACTACTGT CCATTGTCGT GGTTGGTGGT GGGCCTACTG GTGTAGAGGC |
| 1101 | CGCTGGTGAA CTACAGGATT ATGTTCACCA GGACCTGAGA AAGTTTCTCC |
| 1151 | CTGCATTGGC CGAAGAAGTC CAAATTCACT TGGTCGAAGC TCTGCCCATC |
| 1201 | GTTTTGAATA TGTTTGAGAA AAAGCTTTCA TCATACGCGC AATCACATTT |
| 1251 | AGAAAACACT TCGATCAAAG TACATCTGAG AACGGCTGTC GCCAAAGTTG |
| 1301 | AAGAAAAGCA ATTGTTGGCA AAGACCAAAC ACGAAGACGG TAAAATAACC |
| 1351 | GAAGAAACTA TTCCATACGG TACTTTGATT TGGGCCACGG GTAACAAGGC |
| 1401 | AAGACCGGTA ATCACTGACC TTTTCAAGAA AATTCCTGAG CAAAACTCGT |
| 1451 | CCAAGAGAGG ATTGGCAGTG AATGACTTTT TGCAGGTGAA AGGCAGCAAC |
| 1501 | AACATTTTCG CCATTGGTGA CAATGCATTT GCTGGGTTGC CACCAACCGC |
| 1551 | CCAAGTAGCG CACCAAGAGG CCGAATATTT GGCCAAGAAT TTTGATAAAA |
| 1601 | TGGCTCAAAT ACCAAATTTC CAAAAGAATC TATCTTCAAG AAAGGATAAA |
| 1651 | ATTGATCTCT TGTTCGAGGA GAACAACTTT AAACCTTTCA AATACAACGA |
| 1701 | TTTAGGTGCC TTAGCATACC TGGGATCCGA AAGGGCCATT GCAACCATAC |
| 1751 | GTTCCGGTAA GAGAACATTT TACACCGGTG GTGGCTTAAT GACCTTCTAC |
| 1801 | TTATGGAGAA TTTTGTACTT GTCCATGATT CTATCTGCAA GATCGAGATT |
| 1851 | AAAGGTCTTT TTCGACTGGA TTAAATTAGC ATTTTTCAAA AGAGACTTTT |
| 1901 | TTAAAGGATT ATAGATGAAA TTAACATGCC CTTTTCTGGA AAAAGGAAAA |
| 1951 | AAGGTGGTAG GCACCAGTTT TTTCCTGAGT TTGCATCCTT TTTTTTCTAA |
| 2001 | AACCCTCTAA ACAAAACCTA ACACACACAC ACACGCACAA AAAAATGCAC |
| 2051 | ATGATGTTTT ATTATTTATA TATTCCCACT TTTTTCGAAA TGATGCTTGA |
| 2101 | G |

The amino acid sequence of the translated TAT-fusion is:

M R G S H H H H H H G M A S M T G G Q Q
M G R D L Y D D D D K D R W G S K L G Y
G R K K R R Q R R R G G S T M S G Y P Y
D V P D Y A G S M G A G T S F I T S S N
Y T F T Q E K K L K T T M L S K N L Y S
N K R L L T S T N T L V R F A S T R S T
G V E N S G A G P T S F K T M K V I D P
Q H S D K P N V L I L G S G W G A I S F
L K H I D T K K Y N V S I I S P R S Y F
L F T P L L P S A P V G T V D E K S I I
E P I V N F A L K K K G N V T Y Y E A E
A T S I N P D R N T V T I K S L S A V S
Q L Y Q P E N H L G L H Q A E P A E I K
Y D Y L I S A V G A E P N T F G I P G V
T D Y G H F L K E I P N S L E I R R T F
A A N L E K A N L L P K G D P E R R R L
L S I V V V G G G P T G V E A A G E L Q
D Y V H Q D L R K F L P A L A E E V Q I
H L V E A L P I V L N M F E K K L S S Y
A Q S H L E N T S I K V H L R T A V A K
V E E K Q L L A K T K H E D G K I T E E
T I P Y G T L I W A T G N K A R P V I T
D L F K K I P E Q N S S K R G L A V N D
F L Q V K G S N N I F A I G D N A F A G
L P P T A Q V A H Q E A E Y L A K N F D
K M A Q I P N F Q K N L S S R K D K I D
L L F E E N N F K P F K Y N D L G A L A
Y L G S E R A I A T I R S G K R T F Y T
G G G L M T F Y L W R I L Y L S M I L S
A R S R L K V F F D W I K L A F F K R D
F F K G L Stop

We have also demonstrated its efficacy in the isolated perfused heart subjected to ischemia/reperfusion.

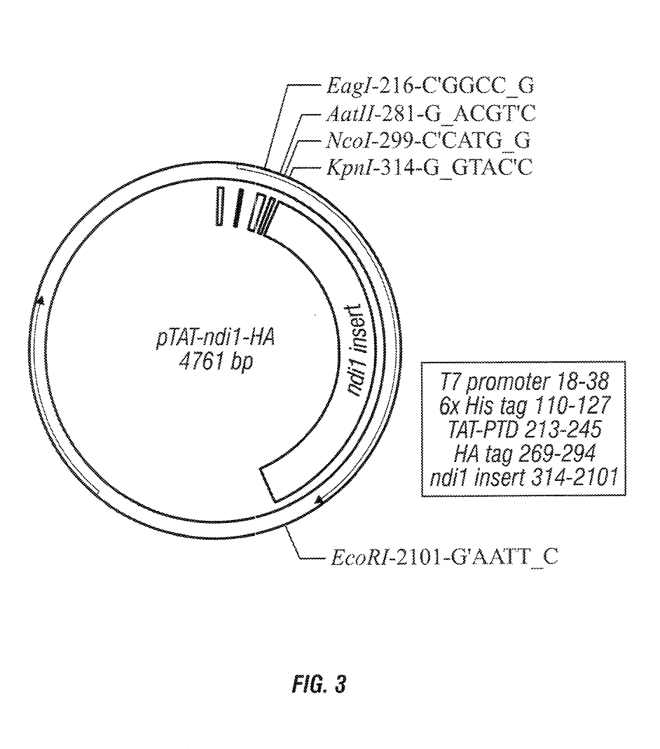

FIG. 3, lower panel, graphically illustrates data demonstrating that delivery of the exemplary chimeric polypeptide of the invention TAT-NDI1 to cells protects them against simulated ischemia/reperfusion (sI/R). FIG. 3, upper panel, illustrates the results of immunostaining with anti-NDI1 antibody (and an anti-cytochrome c antibody); this staining reveals a mitochondrial distribution of TAT-NDI1 that co-localizes with cytochrome c. The three images in FIG. 3, upper panel, include staining with anti-NDI1 antibody only, staining with anti-cytochrome c antibody only, and a merged image. TAT-Ndi1 was added to cardiomyocyte-derived cell line HL-1 cells for 1 hour followed by simulated ischemia reperfusion by exchanging complete Claycomb media with ischemic buffer and placing the cells in a hypoxia chamber for 2 hours. Following a 24 hour reperfusion period cell survival is determined with YoPro1 staining or cells were fixed and stained for Cytochrome C and Ndi1 to demonstrate localization of Ndi1 to the mitochondria. Immunostaining with anti-NDI1 antibody reveals a mitochondrial distribution that co-localizes with cytochrome C. This demonstrates the ability of TAT-Ndi1 to transverse the cell membrane and correctly target to the mitochondria.

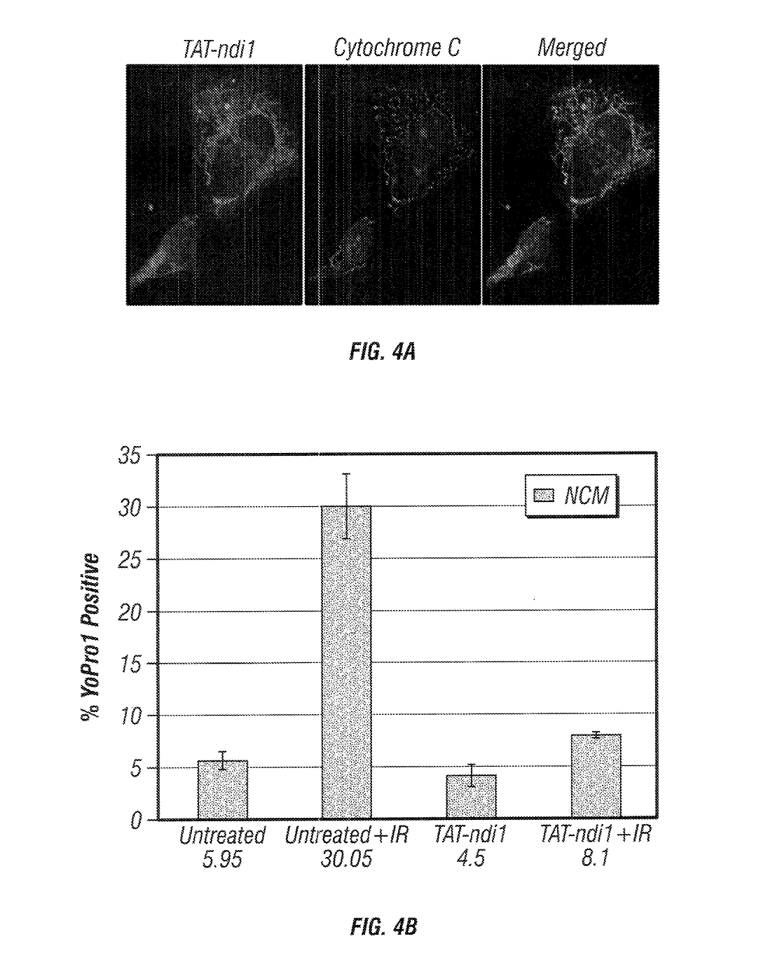

FIG. 4 illustrates data from studies where TAT-NDI1 was purified from bacteria and introduced into the Langendorff-perfused heart by adding to the perfusion buffer for 20 min. Hearts were then subjected to 30 min global ischemia and 2 hr reperfusion. Infarct size was determined by TTC staining and calculation of necrotic area as a percentage of total area. The reduction in infarct size in TAT-Ndi1 perfused hearts as compared to hearts without TAT-Ndi1 demonstrates a cardioprotective capacity of Ndi1 in ischemia reperfusion injury.

FIG. 4 lower right panel graphically illustrates data showing the infarct size (calculation of necrotic area as a percentage of total area) with (“NDI1”) and without (“UNT”) administration of the exemplary chimeric TAT-NDI1 polypeptide of the invention; FIG. 4 lower left panel illustrates TTC staining of necrotic areas with (“NDI1”) and without (“UNT”) administration of the exemplary chimeric TAT-NDI1; and FIG. 4 upper panel graphically illustrates the protocol and timing of this study.

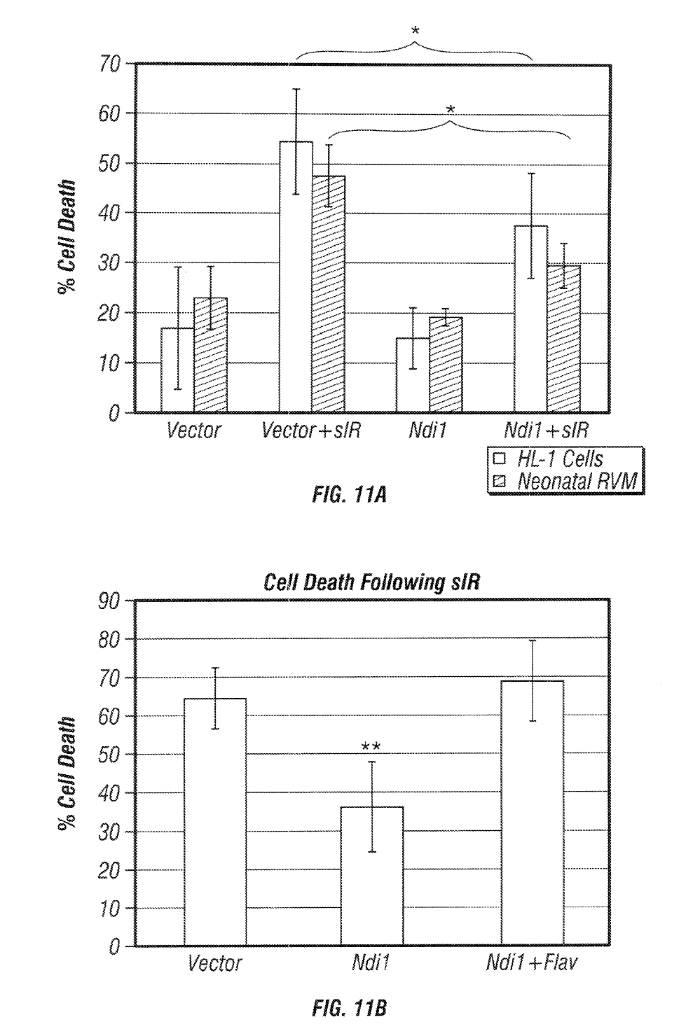

FIG. 11 graphically illustrates data demonstrating that administration of a yeast Ndi1 polypeptide is cytoprotective in cardiomyocytes. To evaluate the ability of Ndi1 to protect against ischemia-reperfusion injury, we initially decided to express Ndi1 in cell culture. The full length NDI1 gene (1,539 bp) was inserted into the pHOOK-2™ vector (Invitrogen, Carlsbad, Calif.) in which expression is driven by the CMV promoter, as described e.g. by Seo (1998) Proc. Natl. Acad. Sci. USA 95:9167-71). The cardiomyocyte derived cell line, HL-1 cells, or neonatal cardiomyocytes were transiently transfected with Ndi1 or empty pHOOK-2™ vector and 36 hours later subjected to simulated ischemia-reperfusion (sIR). Ischemia was induced by buffer exchange to ischemia-mimetic solution (in mM: 125 NaCl, 8 KCl, 1.2 KH₂PO₄, 1.25 MgSO₄, 1.2 CaCl₂, 6.25 NaHCO₃, 5 Na-lactate, 20 HEPES, pH 6.6) and placing the dishes in hypoxic pouches (GASPAK™ EZ, BD Biosciences). After 2 h of ischemia for HL-1 cells and neonatal cardiac myocytes, reperfusion was initiated by buffer exchange to normoxic Krebs-Henseleit solution and incubation at 95% O₂-5% CO₂ for 24 hours. In both neonatal and HL-1 myocytes, Ndi1 provided protection from sIR induced cell death. This protection was determined to be a specific effect of Ndi1 as protection was abolished by the addition of flavone, a specific inhibitor of Ndi1 (data not shown).

In FIG. 11, following simulated ischemia reperfusion, HL-1 or neonatal myocytes expressing Ndi1 or control pHOOK™ vector were scored for cell death by YoPro1 staining. Data expressed as percentage of total cells scored. Ndi1 reduced the percentage of YoPro1 positive cells in comparison to control following simulated ischemia-reperfusion, or sIR. (pValue=<0.05). Ndi1 protected against sIR-induced cell death.

We next decided to evaluate the ability of Ndi1 to protect against IR injury in Langendorff-perfused rat hearts. In order to express the protein in tissue, we generated a TAT-Ndi1 fusion (or chimeric) protein of the invention. Linkage of a minimal 11 amino acid protein transduction domain from HIV TAT is sufficient to transduce a protein into cells in the heart, as described e.g. by Gustafsson (2005) Methods Mol. Med. 112:81-90. The 6×His-TAT-HA cloning vector (pTAT-HA, where HA is hemagglutinin) was provided by Dr Steven Dowdy, Washington University, St Louis, Mo., and is described e.g., in Becker-Hapak (2001) Methods 24:247-256. TAT-NDI1 fusions were generated by insertion of the S. cerevisiae NDI1 open reading frame DNA into the pTAT-HA plasmid and recombinant TAT-fusion protein expression and purification were performed essentially as described by Becker-Hapak (2001) Methods 24:247-256. FIG. 2 schematically illustrates generation of the pTAT-NDI1 plasmid of the invention; the pTAT-HA vector contains an ampicillin resistance marker for selection after transformation, a T7 polymerase promoter, an N-terminal 6-histidine leader before the TAT domain, and an HA tag.

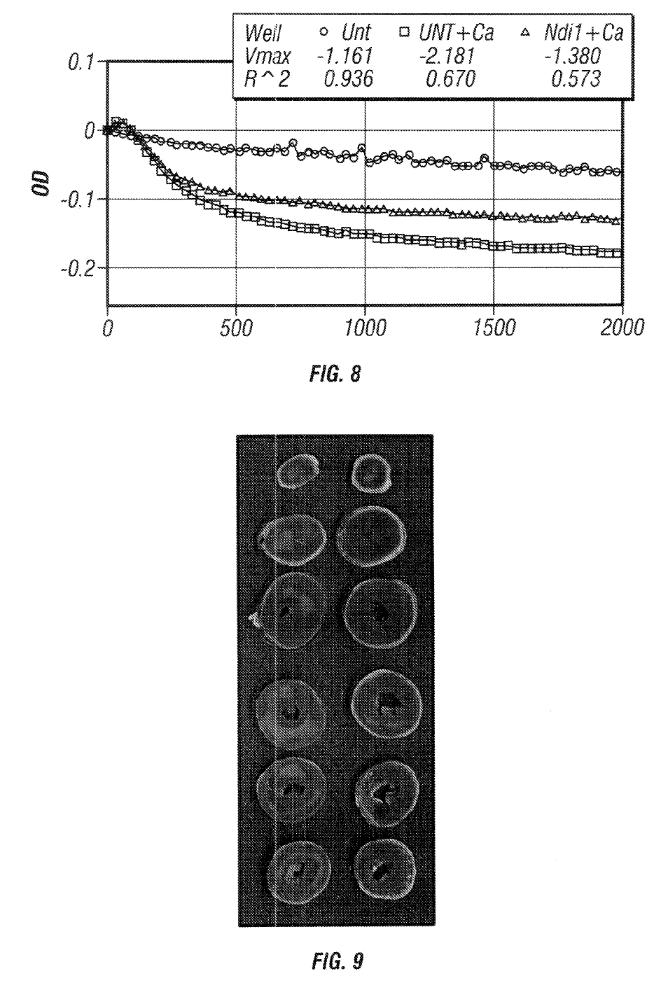

TAT-Ndi1 was added to cultured HL-1 myocytes to confirm localization and functionality of this exemplary fusion protein of the invention. TAT-Ndi1 correctly localized to the mitochondria as confirmed by co-localization with immunofluorescence staining for cytochrome c. Transduced TAT-Ndi1 was also able to protect cultured HL-1 cells from simulated ischemia-reperfusion (sIR)-induced cell death; reducing the level of YoPro1 positive cells to near baseline levels. FIG. 8 schematically and graphically illustrates data demonstrating studies administering an exemplary chimeric (or fusion) Ndi1 polypeptide of this invention; TAT-Ndi1 co-localized with cytochrome C at the mitochondria in HL-1 myocytes.

As illustrated in FIG. 8A, cryosections of rat hearts perfused with TAT-Ndi1 or with control solution (UNT) stained with α-Ndi1 antibody and α-HA antibody. TAT-Ndi1 was expressed in Langendorff-perfused heart tissue.

FIG. 8B illustrate images of immunostains showing that the exemplary Tat-Ndi1 is taken up into cardiomyocytes after perfusion into the isolated perfused heart. First (left) panel is control heart (no Tat-Ndi1 perfusion), stained with anti-Ndi1 antibody and FITC-conjugated secondary antibody. Second (middle) panel is heart that was perfused with Tat-Ndi1 and immunostained with anti-Ndi1 antibody (and FITC-conjugated secondary antibody). Third panel (right) is heart perfused with Tat-Ndi1 and stained with anti-HA antibody (and FITC-conjugated secondary antibody) (HA is an epitope that is contained in the Tat-Ndi1 recombinant protein).

FIG. 9 schematically and graphically illustrates data from studies demonstrating that the exemplary TAT-Ndi1 prevents cell death in HL-1 myocytes subjected to simulated ischemia/reperfusion (sI/R) and hearts subjected to global no-flow ischemia and reperfusion. For the data illustrated in the graph of FIG. 9B (see also left panel FIG. 9D), HL-1 myocytes transduced with TAT-Ndi1 were untreated or subjected to sIR; apoptotic cells were determined by YoPro1 staining. “Unt” is untreated; “I/R” is untreated plus simulated ischemia-reperfusion, or sIR; “Ndi1” is the exemplary TAT-Ndi1 of the invention; “I/R+Ndi1” is IR plus the exemplary TAT-Ndi1 of the invention. The exemplary TAT-Ndi1 of the invention significantly reduced cell death compared to non-expressing cells following sIR.

To determine the ability of TAT-Ndi1 to protect against IR injury, the global ischemia protocol was adapted as described by Tsuchida (1994) Circ. Res. 75:576-585. In brief, the heart was excised from the anesthetized rat and quickly cannulated onto the Langendorff perfusion apparatus. The heart was perfused with Krebs-Ringer buffer (with or without 200 nM TAT protein) for 20 minutes before I/R episodes. No-flow ischemia was maintained for 30 minutes and reperfusion was accomplished by restoring flow for 2 hours. The efficacy of these interventions was determined by measuring infarct size by 2,3,5-triphenyltetrazolium chloride (TTC) staining. We found that TAT-Ndi1 significantly reduced infarct size in this model. The final measurement of cardioprotection will be to measure creatine kinase release which will be completed in the upcoming months.

FIG. 9A (see also the upper right panel of FIG. 9D) illustrates TTC stained heart sections from TAT-Ndi1 (bottom panel FIG. 9A) or unperfused hearts (top panel FIG. 9A) following 30 min global ischemia and 2 hr reperfusion.

FIG. 9C (see also the lower right panel of FIG. 9D) graphically illustrates data quantifying infarct size as % of total tissue. TAT-Ndi1 reduced infarct size from ˜45 to 18% (n=4). The exemplary TAT-Ndi1 was protective against ischemia-reperfusion (IR) injury.

In HL-1 myocytes subjected to 2 hours simulated ischemia and 24 hours reperfusion, superoxide production was increased, NADH accumulated and ATP production was deficient. Expression of Ndi1 was sufficient to significantly reduce ROS levels, as measured by CM-H₂DCFDA (Cat C6827, Molecular Probes) and maintain ATP levels near baseline despite the fact that the Ndi1 enzyme itself does not pump protons. While the invention is not limited by any particular mechanism of action, the preserved ATP levels are most likely attributed to Ndi1's ability to prevent cell death rather than directly increasing ATP production; NADH oxidation, on the other hand, was directly increased by Ndi1 expression.

Ndi1 was transiently transfected into HL-1 myocytes subjected to sIR. ROS levels were measured by CM-H₂DCFDA fluorescence and are expressed in relative fluorescent units (RFU). ATP levels and NAD⁺/NADH ratios were both increased following sIR; representative results are shown in FIG. 14, FIG. 15 and FIG. 16. Flavone, a specific inhibitor of Ndi1, prevented NADH oxidation. Ndi1 restored complex I function in IR.

Measurement of NAD⁺/NADH ratios in Ndi1 expressing cells were an average of 5-fold higher (n=3) compared to pHook2 or untransfected cells following sIR. This was a specific effect since the addition of the Ndi1 inhibitor, Flavone, reversed the NAD⁺/NADH ratio, indicating NADH failed to be oxidized and accumulated in the cells.

These studies were repeated in the ex vivo model of IR with Langendorff-perfused rat hearts. Hearts perfused with TAT-Ndi1 for 20 min, followed by 30 min global, no-flow ischemia and 15 min reperfusion had higher ATP levels and reduced production of ROS as measured by dihydroethidium (DHE) oxidation in stained tissue sections. NAD⁺/NADH ratios will be determined next to see if Ndi1 is able to prevent NADH accumulation caused by CxI inhibition in IR.

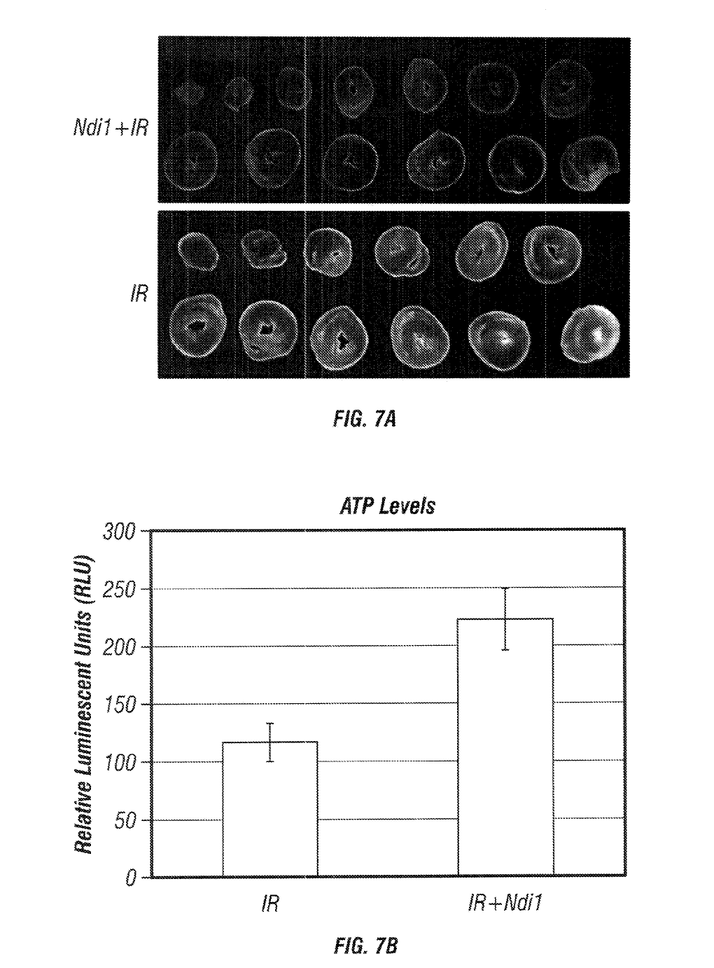

FIG. 2 illustrates data from a study where isolated rat hearts were perfused in Langendorff mode and Tat-Ndi1 or vehicle (control) was introduced into the perfusate for 15 min, followed by 10 min washout. Hearts were subjected to 30 min global no-flow ischemia and 30 min reperfusion. For superoxide production, as illustrated in FIG. 7A, hearts were frozen, then sliced and stained with dihydroethidium; “Tat-Ndi1+IR” is the upper panel and the vehicle control (“IR”) the lower panel—as noted above both samples were subjected to IR; and data is graphically illustrated in FIG. 2B. Superoxide converts dihydroethidium to the fluorescent ethidium product (brighter fluorescence). For ATP measurement, hearts were snap-frozen, then nucleotides were extracted and measured using a luciferase assay. Tat Ndi1 reduced oxidative stress and preserved ATP levels.

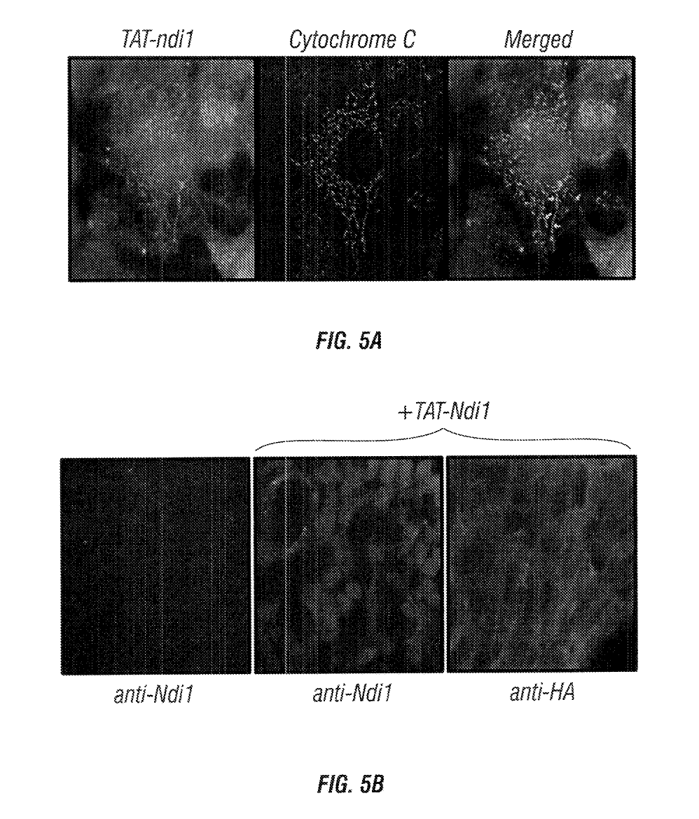

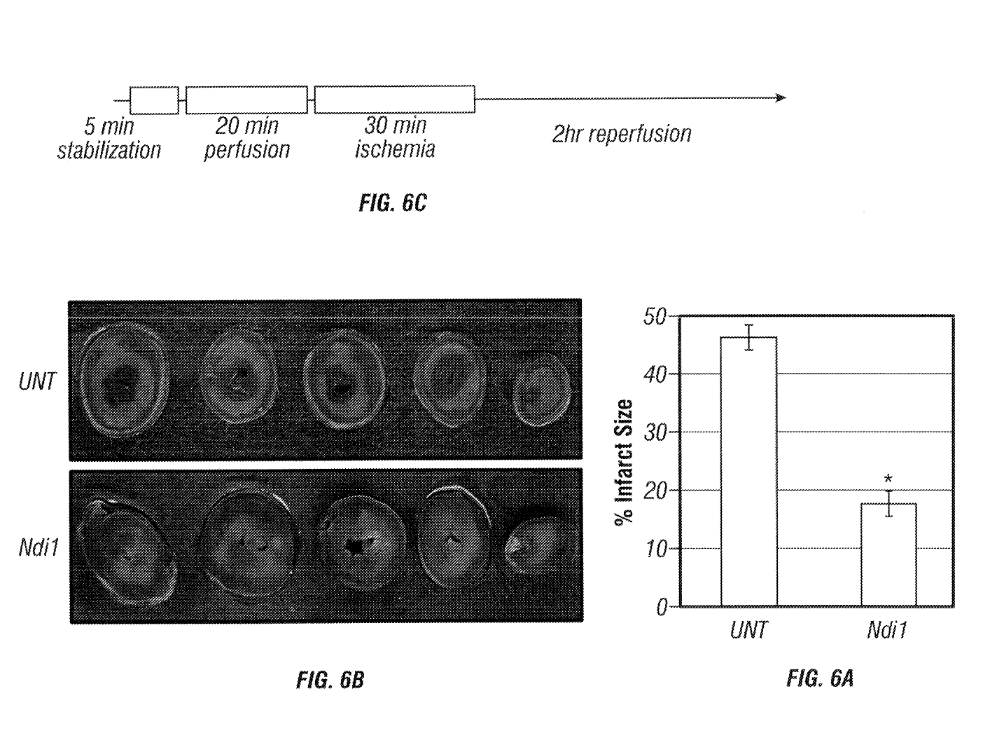

FIG. 5 graphically illustrates spectrophotometry data from a study where isolated rat hearts were perfused with or without Tat-Ndi1, then mitochondria were isolated by polytron homogenization and differential sedimentation. Mitochondria were resuspended under energized conditions and 50 microM (μM) Ca⁺⁺ was added to trigger opening of the mitochondrial permeability transition pore. Swelling was monitored in a plate reader spectro-photometer. Tat-Ndi1 limited the amount of swelling induced by calcium. Ndi1 protected against mitochondrial swelling.

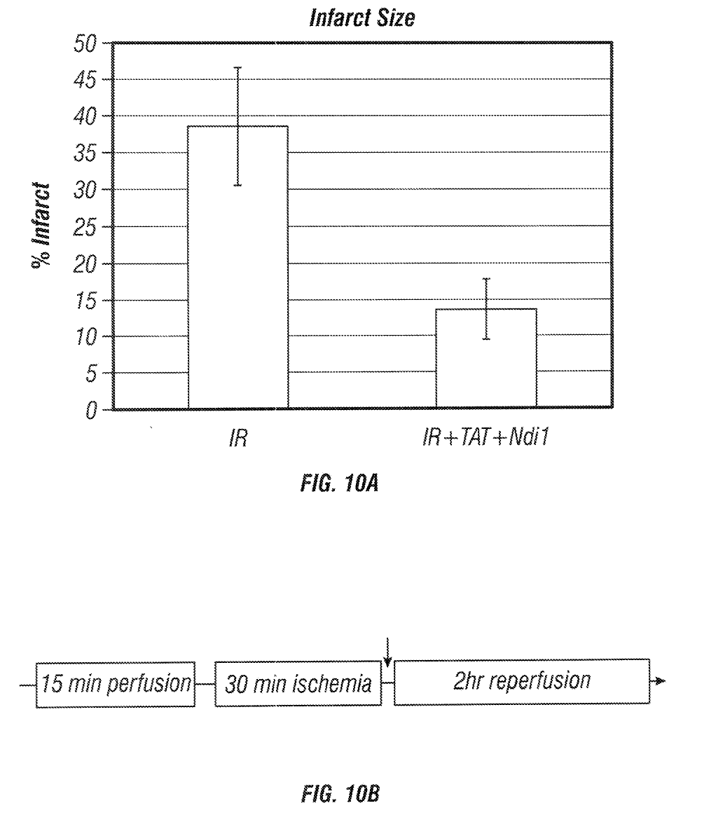

FIGS. 9 and 10 illustrate and summarize data demonstrating Ndi1 as a therapeutic/prophylactic agent. Since it is difficult to predict an acute myocardial infarction before it happens, compositions of the invention are useful as therapeutic/prophylactic agents effective when administered after ischemia—at the time of reperfusion. Isolated rat hearts were perfused in Langendorff mode, then subjected to 30 min global no-flow ischemia; tissue sections are illustrated in FIG. 9. Tat Ndi1 was added to the perfusion buffer at the onset of reperfusion, and infarct size was determined after 2 hr reperfusion. FIG. 13A illustrates a bar graph of data showing the reduction in infarct size (n=2); FIG. 13B illustrates the protocol for this study.

A number of embodiments of the invention have been described. Nevertheless, it will be understood that various modifications may be made without departing from the spirit and scope of the invention. Accordingly, other embodiments are within the scope of the following claims.

1. A chimeric (or fusion) isolated, synthetic or recombinant polypeptide having an NADH oxidoreductase activity, comprising:
(a) (i) a first domain or moiety comprising an NDI1 polypeptide having an NADH oxidoreductase activity, and (ii) at least a second domain or moiety comprising a polypeptide or a peptide;
(b) the chimeric polypeptide of (a)(i), wherein the NDI1 polypeptide comprises or consists of a eukaryotic, a yeast, a Saccharomyces cerevisiae or a human NDI1 polypeptide;
(c) the chimeric polypeptide of (a)(i), wherein the NDI1 polypeptide comprises or consists of an amino acid sequence as set forth in SEQ ID NO:1;
(d) the chimeric polypeptide of any of (a) to (c), wherein the at least a second polypeptide domain or moiety comprises a TAT protein, a taurine, a biotin or a carnitine, or a cell or organelle targeting agent, or a mitochondrial targeting agent, or a carbohydrate-binding domain;
(e) the chimeric polypeptide of any of (a) to (d), wherein the at least a second polypeptide domain or moiety is located amino terminal, carboxy terminal, or amino terminal and carboxy terminal to the NDI1 polypeptide;
(f) the chimeric polypeptide of any of (a) to (e), further comprising a cationic moiety, or a cationic amino acid moiety, or a poly-arginine amino acid residue moiety, or equivalent;
(g) the chimeric polypeptide of (f), wherein the cationic moiety is located amino terminal, carboxy terminal, or amino terminal and carboxy terminal to the NDI1 polypeptide;
(h) a peptidomimetic of the chimeric polypeptide of any of (a) to (g); or
(i) the chimeric polypeptide of any of (a) to (g), or peptidomimetic of (h), further comprising, or modified by: acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation and/or arginylation;
(j) the chimeric polypeptide of any of (a) to (g), or peptidomimetic of (h), wherein the first domain or moiety is joined to the at least second domain or moiety by a chemical linking agent.

2. (canceled)

3. A chimeric (or fusion) isolated, synthetic or recombinant nucleic acid encoding the chimeric (or fusion) polypeptide of claim 1.

4. A composition comprising (a) a first composition comprising the chimeric (or fusion) protein of claim 1, or peptidomimetic form thereof; and a second composition; or (b) the composition of (a), wherein the second composition comprises a liquid, a lipid or a powder.

5. A liposome comprising (a) the chimeric (or fusion) protein of claim 1, or a peptidomimetic form thereof; or (b) the liposome of (a), wherein the liposome is formulated with a pharmaceutically acceptable excipient.

6. A pharmaceutical composition comprising: the chimeric (or fusion) protein of claim 1, or peptidomimetic form thereof; and, a pharmaceutically acceptable excipient.

7. An inhalant or spray formulation comprising: the chimeric (or fusion) protein of claim 1, or a peptidomimetic form thereof; and, a pharmaceutically acceptable excipient.

8. A parenteral or enteral formulation, or a formulation for intrathecal administration or parenteral administration into a perispinal space, comprising: the chimeric (or fusion) protein of claim 1, or a peptidomimetic form thereof and, a pharmaceutically acceptable excipient.

9-10. (canceled)

11. A method for treating, ameliorating or preventing a disease or a condition caused by or aggravated by lost and/or impaired mitochondrial Complex I function, in an individual in need thereof, comprising:
(A)(a) providing the chimeric (or fusion) protein of claim 1, or a peptidomimetic form thereof, or the liposomal form thereof, or the pharmaceutical composition thereof, the inhalant or spray formulation thereof, the parenteral formulation thereof, the enteral formulation thereof, or the intrathecal or perispinal formulation thereof; and
(b) administering an effective amount of (a) to the individual, thereby treating, ameliorating or preventing the disease or condition;
(B) the method of (A), wherein the disease or a condition caused by or aggravated by lost and/or impaired mitochondrial Complex I function is an ischemia and/or reperfusion injury, Parkinson's disease, a myopathic disease, cardiolipin deficiency, a neurodegenerative disease, aging, diabetes, obesity, sepsis or any other condition in which mitochondrial Complex I function is lost and/or impaired;
(C) the method of (A), wherein the disease or a condition caused by or aggravated by lost and/or impaired mitochondrial Complex I function is comprises treatment of mitochondrial dysfunction after myocardial infarction or in heart failure;
(D) the method of (A), wherein the disease or a condition caused by or aggravated by lost and/or impaired mitochondrial Complex I function is as a treatment or pharmaceutical used for organ preservation for transplantation; or
(E) the method of any of (A) to (D), wherein the chimeric (or fusion) protein, peptidomimetic, liposome, pharmaceutical composition, inhalant or spray formulation, parenteral formulation, enteral formulation, or intrathecal or perispinal formulation, is administered after an ischemic event in a heart or other organ, and/or with reperfusion of the heart or other organ.

12. (canceled)

13. An isolated, synthetic or recombinant nucleic acid comprising or consisting of:
(a) a nucleic acid sequence encoding the chimeric (or fusion) polypeptide of claim 1;
(b) the nucleic acid sequence of (a), and further comprising or consisting of nucleic acid sequence encoding a polypeptide antigen, label or tag; or
(c) the nucleic acid sequence of (b), wherein the polypeptide antigen, label or tag comprises or consists of a fluorescent or a detectable protein, or an enzyme, or an enzyme that generates a detectable agent or moiety.

14. A vector, a cloning or expression vector, an expression cassette, a plasmid, a phage, or a recombinant virus, comprising the isolated or recombinant nucleic acid of claim 13.

15. A host cell comprising
(a) the nucleic acid of claim 3; or
(b) the host cell of (a), wherein the cell is a bacterial cell, a mammalian cell, a fungal cell, an insect cell, a yeast cell or a plant cell.

16. A non-human transgenic animal comprising
(a) the nucleic acid of claim 1; or
(b) the non-human transgenic animal of (a), wherein the animal is a mouse or a rat.

17. (canceled)

18. An inhaler, nebulizer or atomizer comprising the chimeric (or fusion) protein of claim 1, or a peptidomimetic form thereof.

19. A pharmaceutical composition comprising
(a) the chimeric (or fusion) protein of claim 1, or a peptidomimetic form thereof, or any combination thereof; or
(b) the pharmaceutical composition of (a), further comprising a pharmaceutically acceptable excipient.

20-25. (canceled)


SEQUENCE LISTING (3 sequences)

SEQ ID NO: 1 - protein, 513 aa, Saccharomyces cerevisiae
MLSKNLYSNKRLLTSTNTLVRFASTRSTGVENSGAGPTSFKTMKVIDPQHSDKPNVLILGSGWGAISFLKHIDTKKYNVSIISPRSYFLFTPLLPSAPVGTVDEKSIIEPIVNFALKKKGNVTYYEAEATSINPDRNTVTIKSLSAVSQLYQPENHLGLHQAEPAEIKYDYLISAVGAEPNTFGIPGVTDYGHFLKEIPNSLEIRRTFAANLEKANLLPKGDPERRRLLSIVVVGGGPTGVEAAGELQDYVHQDLRKFLPALAEEVQIHLVEALPIVLNMFEKKLSSYAQSHLENTSIKVHLRTAVAKVEEKQLLAKTKHEDGKITEETIPYGTLIWATGNKARPVITDLFKKIPEQNSSKRGLAVNDFLQVKGSNNIFAIGDNAFAGLPPTAQVAHQEAEYLAKNFDKMAQIPNFQKNLSSRKDKIDLLFEENNFKPFKYNDLGALAYLGSERAIATIRSGKRTFYTGGGLMTFYLWRILYLSMILSARSRLKVFFDWIKLAFFKRDFFKGL

SEQ ID NO: 2 - DNA, 1791 bp, artificial sequence
  synthetic nucleic acid
gtaccagtttcatcacatcatcgaattacacgtttacccaagaaaagaaactaaaaaccactatgctatcgaagaatttgtatagtaacaagaggttgctcacctcgacgaatacgctagtcagattcgcttccaccagatccacaggggtggaaaactccggagcaggtcctacatcttttaagaccatgaaagtcattgaccctcagcacagcgacaaaccaaacgtgctgatactgggttcggggtggggagctatttcgtttttaaagcacattgacaccaagaagtacaacgtttccatcatctctcctagaagctatttcttatttacgcctttgttaccttctgcaccagttgggacagtagacgaaaagtcaattattgagcccatcgttaattttgctctcaagaaaaaggggaacgttacctactatgaggcagaagccacctctatcaatcccgacaggaataccgttaccataaaatcattatctgccgttagccagctataccaacctgaaaaccatctagggctgcatcaagcagaacctgctgaaattaagtacgattatttaatcagtgctgtaggtgcggaacctaacacatttggtattcctggggtcactgattacggtcatttcctgaaggaaattcccaactctttggaaataagaagaacttttgccgccaatctagagaaggctaacttattgccaaagggtgatcccgaaagaagaagactactgtccattgtcgtggttggtggtgggcctactggtgtagaggccgctggtgaactacaggattatgttcaccaggacctgagaaagtttctccctgcattggccgaagaagtccaaattcacttggtcgaagctctgcccatcgttttgaatatgtttgagaaaaagctttcatcatacgcgcaatcacatttagaaaacacttcgatcaaagtacatctgagaacggctgtcgccaaagttgaagaaaagcaattgttggcaaagaccaaacacgaagacggtaaaataaccgaagaaactattccatacggtactttgatttgggccacgggtaacaaggcaagaccggtaatcactgaccttttcaagaaaattcctgagcaaaactcgtccaagagaggattggcagtgaatgactttttgcaggtgaaaggcagcaacaacattttcgccattggtgacaatgcatttgctgggttgccaccaaccgcccaagtagcgcaccaagaggccgaatatttggccaagaattttgataaaatggctcaaataccaaatttccaaaagaatctatcttcaagaaaggataaaattgatctcttgttcgaggagaacaactttaaacctttcaaatacaacgatttaggtgccttagcatacctgggatccgaaagggccattgcaaccatacgttccggtaagagaacattttacaccggtggtggcttaatgaccttctacttatggagaattttgtacttgtccatgattctatctgcaagatcgagattaaaggtctttttcgactggattaaattagcatttttcaaaagagacttttttaaaggattatagatgaaattaacatgcccttttctggaaaaaggaaaaaaggtggtaggcaccagttttttcctgagtttgcatccttttttttctaaaaccctctaaacaaaacctaacacacacacacacgcacaaaaaaatgcacatgatgttttattatttatatattcccacttttttcgaaatgatgcttgag

SEQ ID NO: 3 - protein, 605 aa, artificial sequence
  synthetic polypeptide
MRGSHHHHHHGMASMTGGQQMGRDLYDDDDKDRWGSKLGYGRKKRRQRRRGGSTMSGYPYDVPDYAGSMGAGTSFITSSNYTFTQEKKLKTTMLSKNLYSNKRLLTSTNTLVRFASTRSTGVENSGAGPTSFKTMKVIDPQHSDKPNVLILGSGWGAISFLKHIDTKKYNVSIISPRSYFLFTPLLPSAPVGTVDEKSIIEPIVNFALKKKGNVTYYEAEATSINPDRNTVTIKSLSAVSQLYQPENHLGLHQAEPAEIKYDYLISAVGAEPNTFGIPGVTDYGHFLKEIPNSLEIRRTFAANLEKANLLPKGDPERRRLLSIVVVGGGPTGVEAAGELQDYVHQDLRKFLPALAEEVQIHLVEALPIVLNMFEKKLSSYAQSHLENTSIKVHLRTAVAKVEEKQLLAKTKHEDGKITEETIPYGTLIWATGNKARPVITDLFKKIPEQNSSKRGLAVNDFLQVKGSNNIFAIGDNAFAGLPPTAQVAHQEAEYLAKNFDKMAQIPNFQKNLSSRKDKIDLLFEENNFKPFKYNDLGALAYLGSERAIATIRSGKRTFYTGGGLMTFYLWRILYLSMILSARSRLKVFFDWIKLAFFKRDFFKGL